(19) United States
(12) Patent Application Publication
(10) Pub. No.: US 2021/0162154 A1
(43) Pub. Date: Jun. 3, 2021

(54) A Supraglottic Airway Device for intermittent Positive Pressure Ventilation

(71) Applicant: Yang Sun, San Francisco, CA (US)

(72) Inventor: Yang Sun, San Francisco, CA (US)

(21) Appl. No.: 16/699,601

(22) Filed: Nov. 30, 2019

(51) Int. Cl. A61M 16/04

(52) U.S. Cl. CPC A61M 16/045; A61M 16/0447; A61M 1/008; A61M 2210/0656; A61M 2205/0216; A61M 16/0409

(57) ABSTRACT

An airway device to be inserted into a patient's throat and to be function as a supraglottic airway device during a positive pressure ventilation and spontaneous ventilation that includes an air tube, an air sac, a tongue lifting means, a pulling unit and a drainage systems. The pulling unit will be pulled by an operator's hand to cause the tongue lifting means upward movement to open up the air sac and lift up the tongue base. The air tube lumen and the air sac are configured to have inflow airflow be divided and follow specific route therefore to deliver the focused airflow to vocal cords opening, and also to let inspired airflow enter desired area of the air sac to expand the air sac to form seals with surrounding tissue. During expiration, the pressure will be relieved. Different design of the drainage system can be configured to drained fluid from upper esophagus or from posterior pharyngeal wall.

### RELATED APPLICATION

This application is a continuation-in-part of co-pending application Ser. No. 16/163,501. The co-pending application Ser. No. 16/163,501 filed on Oct. 17, 2018 entitled “A Supraglottic Airway Device for Intermittent Positive Pressure Ventilation” in its entirety is hereby incorporated by reference. Claims 1-15 are based on the Ser. No. 16/163,501 patent application and the remaining claims are based on new materials within this application.

### FIELD OF THE INVENTION

The present invention relates to medical airway management devices and methods that can be used as an independent supraglottic airway device for positive pressure mechanical ventilation and spontaneous ventilation to replace traditional endotracheal intubation in many cases.

### BACKGROUND OF THE INVENTION

It is standard practice of requiring endotracheal intubation on a patient when positive mechanical ventilation is planned. Using different type of supraglottic airway devices such as different type of LMAs, I-gel, air-Qsp, Baska mask, laryngeal tube and others for spontaneous ventilation has been widely accepted, but not for mechanical ventilation which can result in significant air leakage to make positive pressure mechanic ventilation ineffective and has other complications.

The present invention is intended to improve effectiveness and safety of supraglottic ventilation without using an endotracheal intubation for many currently intubated cases and also to be used for spontaneous ventilation. The present invention is easy to use and can be used in multiple medical specialties without waiting for a sub-specialty trained physician in many emergency airway cases.

### SUMMARY OF THE INVENTION

The device may be constructed from any suitable plastics, silicones, rubbers polymers, or other suitable materials or combinations with sufficient flexibility that allows insertion into a patient's throat without damaging throat tissues. The present invention can be sized to accommodate different gender and age group of patient.

As will be realized, the invention is capable of other and different embodiment and its several details are capable of modification in various respects, while still attaining the beneficial results of the present invention all without departing form the invention. These components can be modified in different configurations and combinations, and re-arrangement of the basic components, or omission of some components of this invention, or these components can be made of different type of materials. However they are still within the scope of this invention. Accordingly, the drawings and descriptions are to be regarded as illustration to nature of the device and not in a restrictive or limitation sense.

For purpose of description, the use the word of “distal” refers to the device's end that first enters into a patient's mouth. The term “proximal” is the opposite end. The term “patient” refers to any person or animal requiring the use of the device. The use of word “posterior” refers to a back side of the device or a patient or animal. The word “anterior” is the opposite side. Further, the use of the term “communication” is used to mean a path for air, gas, or fluid to flow. For purpose of easy describing, if not otherwise specified, the device is assumed in a semi-horizontal or diagonal position as illustrated in FIG. 1 with the air sac opening toward upside and the air sac bottom wall toward the downside. Therefore when tongue lifting plate is pulled up by pulling band, it will be described as “pulled up, moving up, rising” and so on. And if the device has inserted into a patient's throat, it will be assumed that patient or animal is in supine position unless specified otherwise.

Please note, term “couple” or “attach to” between different parts include but not limited to the use of glue, thermo-adhesive, fasteners or other suitable techniques, or combination thereof or any other suitable technologies.

The present invention can be sized to accommodate different gender and age group of human and selected groups of animals. All components are made with biocompatible materials. These include but not limited to plastics, silicones, polymers, rubbers, metals, a combination thereof, or a combination with other types of materials.

The following three embodiments can be derived from the same basic principle of this invention.

The first embodiment: comprises an air tube, an air sac, a tongue lifting means, and a pulling unit 60 with optional one or more drainage tubes.

In first embodiment the air tube outside shape is different with its inside shape. The drainage passage 100 is configured to “invade” inside air tube lumen 12 at Y point and couple to the air tube middle bottom wall longitudinally therefore to divide or reshape the air tube lumen into a top tube airflow channel 30, a right tube airflow channel 32 and a left tube airflow channel 31 respectively.

The air tube lumen 12 in cross section view from Y point to the air tube distal opening 29 is a sum of cross sections of all three tube airflow channels.

After passing beneath the air tube distal edge 29, the drainage passage 100 continues with the distal drainage tube 109 to form a sac central protruding surface 39 which includes an upward ramp 22 and a downward ramp 23. The upward ramp can provide guidance for airflow or passing a stylet into laryngeal inlet.

Air sac include air sac bottom wall, a right sac airflow channel 78 and a right sac wall 81, a left sac airflow channel 79 and a right and a left sac wall 81, and a sac distal pocket.

The air sac bottom wall has the sac central protruding surface 39 which is “sandwiched” by a right sac airflow channel 78 on its right side and a left sac airflow channel 79 on its left side. The right and left sac wall include a right and a left holding zone 85 which are bent inward toward each other and couple to the air tube top wall. The right and left sac proximal attachment of the air sac is to attach to the air tube distal segment 15.

The right and left sac airflow channels are smoothly continue proximally with the right and left tube airflow channel respectively and continue to extend distally to form a sac distal pocket 99. The airflow from inside the right or left tube airflow channel will flow into the right and left sac airflow channel 78,79 respectively and continue to flow distally into the sac distal pocket 99. Finally the airflow flow out the sac distal pocket 99 from the beneath of the sac distal pocket cover 89 into the air sac chamber. The airflow in the top tube airflow channel 30 will flow toward the vocal cords opening.

The whole air sac is made of the materials with certain compressibility and elasticity. The shape of the right and left sac airflow channel 78,79 and the sac distal pocket can be compressed during insertion of the device. The flexibility and elasticity also allow the air sac being pulled by a mechanical force and pushed by inspired airflow to open further especially during Intermittent Positive Pressure Ventilation

These channeled airflows are able to push the air sac to form seals with pharyngeal walls or surrounding tissues during inspiration phase of a respiration cycle after the pulling unit 60 pull up the tongue base and open up more the sac chamber. And the pushing force is the inspiratory pressure dependent and is proportion to the inspiratory pressure.

The tongue lifting means can be configured as a lifting plate 51, or a bar lifting assembly 95. The both are pulled by the pulling unit 60.

An operator will have to pull the pulling band tail to lift up tongue lifting means to change physical position of the tongue lifting means before the device can perform its functions.

The lifting plate has a plate distal edge and right and left plate side edges and is couple to the air tube distal edge 29. An indentation line 25 is configured at connection between the lifting plate 51 and the top edge of the air tube distal edge 29 to make the lifting plate easily to be bent. And therefore the lifting plate 51 can use the indentation line 25 as a bending “joint” to form a dull angle with the top wall of the air tube distal end 29 when the lifting plate is pulled up diagonally.

Before the lifting plate 51 is pulled, right and left inward strip 86 of the air sac are toward to each other obliquely. When tongue lifting plate 51 is pulled up diagonally by the pulling bands, the right and left sac wall 81 are pushed further apart by the right and left lifting plate side edge 52, and the right and left inward strip 86 are to be pushed up by the lifted plate distal edge 53. At same time the lifted plate distal edge will form the seal with the tongue base and the raising lifting plate 51 will push right and left the sac wall holding zone 85 toward proximal direction which consequently will stretch the inward strip 86 in proximal direction. Then right and left inward strip 86 will stretch right and left sac wall descending edges 82 to keep the right and left sac airflow channels open to be expanded by incoming inspired airflow and pull the sac distal pocket cover to form a seal with posterior laryngeal wall. And the right and left sac wall descending edges are also able to meet the right and left side structures of the laryngeal inlet therefore to stop further advance the device during insertion.

Each of two epiglottis elevation plates on right and left side of the plate distal edge 53 or only one epiglottis elevation plate epiglottis in the middle of the plate distal edge can been configured to push up the epiglottis to open the laryngeal inlet when the lifting plate 51 is pulled up.

In an alternative design, when the bar lifting assembly 95 is pulled up diagonally the bar lifting assembly will be unfolded to form seal with tongue base. The right and left side fold 96 will also unfold to form seal with the sac wall holding zone 85 and prevent the air escape from the air sac chamber.

The enhanced sealing means has been configured which include the sac sealing belt 90, the right and left plate sealing petals 55, the right and left plate sealing bumps 57, the right and left corner sealing folds 56 and the right and left sac wall holding zone angles 83, all of them are configured to enhance the sealing between the right and left sac wall holding zone 85 and the lifting plate 51.

The pulling unit 60 are one or more bands and are designed to pull up the tongue lifting means, and consequently the tongue lifting means will push up the base of tongue. And the pulling unit 60 can pull up the tongue base independent of action of pulling the tongue lifting means and hold and stabilize the tongue in an elevated position.

The right and left pulling bands 66 pass through the space between the right and left side of the sac wall holding zone and touch the sac wall proximal edges 88. When the right and left pulling bands 66 are pulled and tightened, the pulling bands 66 will push right and left sac wall holding zone further apart.

One or more hooks can be configured on the wall of the air tube or the drainage passage to hold the band hole. Therefore, operator can free a hand while operating the device.

A band sliding pad 65 has been configured to convey an operator's pulling force to right and left band distal ends 68.

In a preferred embodiment, the right or left band distal end 68 separately attach to the right and left side of the tongue lifting means.

In the first embodiment, the drainage passage 100 is configured to include a first drainage tube 101 and a second drainage tube 102. After passing beneath of the air tube distal opening 29 the first drainage tube 101 continues distally with the distal drainage tube 109 with its distal opening positioned under the sac distal pocket to drain the fluid from the upper esophagus.

The function of the second drainage tube 102 is to drain fluid or secretion cumulated under the fluid groove 77.

Alternatively, the drainage passage can be configured as a central drainage tube 111. The central drainage tube 111 divides the air tube lumen and passes beneath the air tube distal end 29 to drain cumulated fluid under the fluid hood 115.

### The Second Embodiment

The second embodiment comprises an air tube, an air sac, a tongue lifting means, a pulling unite and an optional drainage system which includes optional one or more drainage tubes. In addition the divergent plate 28a the distal airflow bottom wall 29a distal airflow tunnel, the band anchor 65a and band stick 66a are newly added structures.

Same as in first embodiment, the air tube outside shape in the second embodiment is changeable and its lumen is reshaped by drainage passage 100 starting at the Y point 18. Therefore the air tube lumen starting from the Y point 18 is divided longitudinally or reshaped into a top tuber airflow channel 30, a right tube airflow channel 32 and a left tube airflow channel 31 respectively. At the split point 18c, a length of the air tube distal segment 15 form its own distal airflow bottom wall 29a and extends further into air sac. This length segment of the air tube distal segment 15 can be configured as either the distal airflow tube 38 or the distal airflow channel 15a. The both have up-curved shape and extend distally close to the vocal cords opening.

In the distal airflow bottom wall 29a is configured to have a central groove 29c to guide a stylet for a tracheal intubation.

Preferably a right and a left divergent plate 28a are configured to divergent more airflow from right and left tube airflow channel 32, 31 into the distal airflow tube 38 or the distal airflow channel 15a and create the pressurized focused or jet like airflow toward the vocal cords opening. The ability of the divergent plate 28a able to be pushed and to change its position automatically according to changes of the airflow pressure inside air tube lumen has created an airflow self-control mechanism which can automatically adjust amount of airflow into the distal airflow tunnel in positive mechanic ventilation.

After passing beneath the split point 18c distally, the first drainage tube 101 of the drainage passage 100 continues with the distal drainage tube 109 which forms a sac central protruding surface 39 same ways as the first embodiment except no the upward ramp 22 and the downward ramp 23 configuration. The rest of the sac bottom wall would be same as in the first embodiment. The air sac in the second embodiment also includes, a right sac airflow channel 78 and a right sac wall 81, a left sac airflow channel 79 and a right and a left sac wall 81, and a sac distal pocket same configurations as in the first embodiment.

The right and left sac wall include a right and a left sac wall holding zone 85 which the most proximal portion of the sac wall and are bent inward toward each other and couple to the air tube top wall same as in the first embodiment.

The tongue lifting means and pulling unite are also configured same the lifting plate 51, or a bar lifting assembly 95 as in the first embodiment. But a sac lifting plate 99a, a new alternative, has been configured to be pulled and to lift tongue base. The all three are pulled by the pulling unit 60 and also use the indentation line 25 as a bending “hinge” or joint” when the lifting plate is pulled up diagonally same as in the first embodiment.

One or more pairs of band anchor 65a have been configured in right and left side of the air tube side wall as alternative of the band sliding pad 65. The pulling band can slide back and forth underneath the band anchor 65a therefore to transfer an operator's pulling force to the right and left band distal ends 68 and to the tongue lifting means.

The rest of structure, such as the enhanced sealing means, the drainage system and alternative design of the central drainage tube 111 can be optionally configured in the second embodiment the same as in the first embodiment.

### Third Embodiment

The third embodiment comprises the same tongue lifting means and the pulling unit But the air tube and air sac have some different modifications.

In the third embodiment, a distal portion of the air tube distal segment 15, called distal airflow segment 118 will extend inside the air sac and curves up same as in second embodiment. On the distal airflow segment 118, one or more air holes 116 holes have been configured on right and left side walls, or bottom wall, or corners of the bottom wall and right and left side walls of the distal airflow segment 118. The air holes 116 are to let a portion of an incoming airflow from the air tube flow into right and left side of the air sac chamber.

A central thinking area 39a in the sac bottom wall is to reinforce the rigidity of the air sac bottom wall and to form the right and left sac airflow channel 118a, 118b. The right and left sac airflow channel in this embodiment will be shallower than the first and second embodiments but still able to accommodate the airflow from the right and left tube airflow channels and able to function same as the right and left sac airflow channel in the first and second embodiments. The airflow will be divergent by a pair or more air holes into right and left sac airflow channel of the air sac 119. Then airflow inside of the right and left sac airflow channel will also flow toward the distal end which will cause the sac distal pocket cover 121 expand therefore to create a seal with posterior laryngeal wall same way as in the first and second embodiments. However in third embodiment, the central thinking area 39a and the right and left sac airflow channel 118a,118b can be omitted therefore the airflow inside the air sac will not be divided.

### BRIEF DESCRIPTION OF THE DRAWINGS

FIG. 1—Left side oblique view of the device 10 of the first embodiment, including the air tube which includes an air tube proximal segment 13 and an air tube distal segment 15, the air sac 80, the lifting plate 51 and the pulling unit 60 an and the drainage passage 100. The lifting plate 51 has not been pulled up and is in a relaxed un -pulled position. For illustration purpose, visible portion of the pulling unit 60 are painted as the darker area and grey area of the pulling unit 60 represent segments which are behind other structures and cannot be directly visualized.

FIG. 2—Left side oblique view of FIG. 1 in the first embodiment, an operator is pulling the band tail 63 of the pulling unit. The lifting plate 51 is being pulled up by an operator. The lifting plate 51 is in pulled up position. The sac top opening 70 is wide open therefore the air tube distal opening 29, the sac central protruding surface 39, the upward ramp 22 and the downward ramp 23 are also shown.

FIG. 2a—Left side oblique view of the second embodiment, an operator is pulling the band tail 63 of the pulling unit. The lifting plate 51 is being pulled up. by an operator same way as in the first embodiment. The sac top opening 70 is wide open. The air tube distal opening 29, central groove 29c and a left distal airflow side wall 29b are shown. A new alternative design of right and left pulling sticks 66a have been replaced the pulling bands 66 and are pulled by an operator same way as in the first embodiment, and are sliding along beneath right and left band anchors 65a.

FIG. 3—Left side view of a cutting-through from the middle of the device in the first embodiment, the pulling bans has been pulled and the lifting plate 51 is pulled up. Please note that the drainage passage top wall 35 is configured to continue with the central longitudinal protruding surface 39 which includes the upward ramp 22 and the downward ramp 23. And the first drainage tube 101 continues after passing beneath the air tube distal end 29 and become the distal drainage tube 109.

FIG. 3a—Left side view of a cutting-through from the middle of the device of the second embodiment, the pulling bands have been pulled and the lifting plate 51 is pulled up. Please note that the drainage passage top wall 35 is configured to continue with the top wall of the distal drainage tube 109 which form the central longitudinal protruding surface 39.

In this draining, right pulling band touch and pass right sac wall proximal edges 88. The distal airflow channel 15a has the incomplete top wall 29d therefore the air tube distal opening 29 is enlarged as shown. The distal airflow bottom wall 29a branch out from the drainage passage top wall 35 and curve up therefore from the split point 18c and the split angle 18b. The left distal airflow side wall 29b is painted as grey area.

FIG. 4—Left side view after the device being inserted into a patient's throat.

FIG. 5—Left posterior oblique view the first embodiment, the pulling bands are not shown in order to better demonstrate the drainage system. The second drainage tube distal opening 108 is terminated at or shortly after passing the split point 18c to drain the left bottom fluid groove 77.

FIG. 6a—Explored view, the air sac has been de-attached from the air tube, viewing from left side of the air sac. Please note that the sac horizontal attachment edge 72 is curved structure and is painted in thicker darker line. The sac wall holding zone 85, inward strips 86, the sac wall descending edges 82, the sac distal pocket cover 89, the sac distal edge 87, and the distal drainage tube 109 are shown.

FIG. 6b Explored oblique distal view after the air sac being detached in the second embodiment , the divided three airflow channels viewed from the distal direction in comparison with the first embodiment in FIG. 6c. The first drainage tube 101, and distal opening of the right and left tube airflow channel 32,31 are shown. The tongue lifting plate 51 has been pulled up by the pulling bands 66, the pulling bands 66 are anchored underneath the band anchor 65a. The inferior surface of the tongue lifting unite now is facing to distal direction and the air tube distal opening 29 is seen. The distal airflow bottom wall 29a and the central groove 29c are shown. The left distal airflow side wall 29b and the incomplete top wall 29 are painted as striped areas.

FIG. 6c—Explored oblique distal view after the air sac being detached in the first embodiment, the divided three airflow channels viewed from the distal direction, the first drainage tube 101 is configured to continue with the distal drainage tube 109, but continuality has be cut in the drawing. The tongue lifting plate 51 has been pulled up by the pulling bands 66. The inferior surface of the tongue lifting unite now is facing to distal direction.

FIG. 7a—Explored view of Left side view of the first drainage tube 101 and the distal drainage tube 109, the first drainage tube is continuing with the distal drainage tube 109, the distal drainage tube 109 has a distal drainage tube distal opening 105. The upward ramp 22 and the downward ramp 23 are configured on the top surface of the sac central protruding surface 39.

FIG. 7b—Posterior view of the drainage system the first drainage tube 101 and the second drainage tube 102, and the distal drainage tube 109. The second drainage tube distal opening 108 terminates early.

FIG. 8a—n oblique view of a new design of the sac lifting plate 99a in the second embodiment from a proximal direction. The sac lifting plate 99a has been pre-manufactured in a semi-horizontal folded toward distally in un-pulled position. The right and left sac walls 81 have been pushed down or folded down toward bottom of the sac chamber. The left the sac airflow channel side wall 76, the pulling bands 66 and the band anchors 65a are shown.

FIG. 8b—An oblique view of the FIG. 8a from a distal direction, an alternative design. The sac lifting plate 99a has been pulled up. The inferior surface of the sac lifting plate 99a is facing distally. The sac lifting plate 99a pulls the inward strip 86 and the sac wall descending edge 82 in tense. The sac walls 81 have also been pulled and stood up. Left sac wall 81 is painted grey area. The sac top opening 70 is wide open therefore the distal airflow tube 38 and the air tube distal opening 29 can be seen.

FIG. 8c—an alternative design of the lifting plate, the bar lifting assembly 95 is configured as “umbrella” like, and is in relaxed un-pulled position. The bar lifting assembly 95 is painted as a grey area and it folded like an “umbrella” inside of the air sac.

FIG. 9a—Oblique view of the an alternative design of the first embodiment, showing connection of the sac proximal attachment couple to the air tube distal segment 15, in which the sac vertical attachment edge 71 couples to the left air tube side wall 16 and the sac horizontal attachment 72 couples to the air tube top wall. The corner sealing fold 56 is shown. The lifting plate 51 has not been being pulled up, but the pulling unit 60 and the drainage tubes are not shown for better illustrating the corner sealing fold.

FIG. 9b—zoom up view of the sac vertical attachment edge 71, the sac horizontal attachment edge 72 and indentation line 25 (as painted double line). The sac horizontal attachment edge 72 and the sac vertical attachment edge 71 couple to the air tube distal segment. The sac horizontal attachment edge 72 curves back distally to reach the indentation line 25 to form the sac wall holding zone angle 83.

FIG. 9c—showing the sac sealing belt 90 as one of the enhanced sealing means. The upper horizontal edge 92 and lower horizontal edge 91 of the sac sealing belt 90 are shown.

FIG. 9d—Left oblique view from back of an alternative design of the first embodiment, no configuration of the second drainage tube 102, and no the distal drainage tube 109. The central drainage tube 111 with its central drainage tube distal opening 113 terminating at the fluid hood 115. The esophageal fluid can enter the fluid hood 115 through the fluid entrance 112.

FIG. 10a—Top view of the lifting plate 51 and the pulling unit 60 in a relaxed un-pulled position. Two epiglottis elevation plates 58 are configured as two protruding structures toward distally. The right and left plate sealing bump 57 are shown.

FIG. 10b—Top view of an alternative design of the pulling unit 60 with one single band distal converged end 69 attach to the lifting plate. The central epiglottis elevation plate 59 is shown.

FIG. 10c—Top view of an alternative of the enhanced sealing means, the right and left plate sealing petal 55, and an alternative design of the pulling unit 60 with a band bridge 64 connecting the right and left band bands 66 are shown. The epiglottis elevation plates 58 are shown.

FIG. 11—Multiple cross-sectional view lines of the device, the pulling unit not shown.

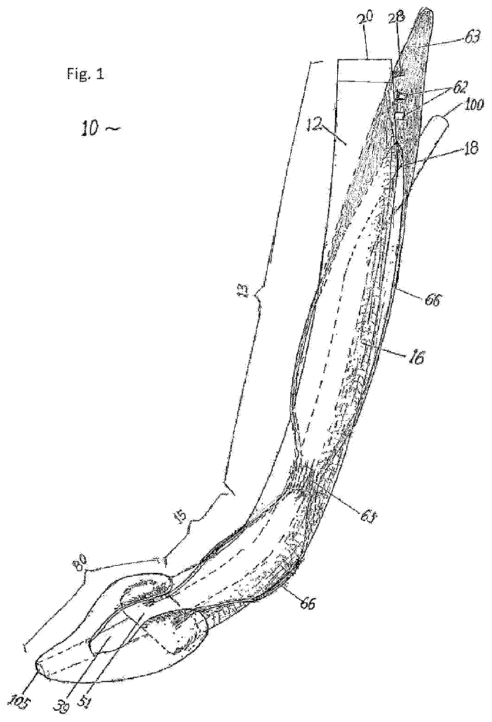

FIG. 1a—A cross sectional view through the sac distal pocket 99.

FIG. 11b1—A cross sectional view through the sac wall descending edge of the air sac. A cross section of the semi-circled semi-opened right and left sac airflow channel are not yet being further opened before the lifting plate is pulled to cause the sac wall descending edge to be pulled and stretched.

FIG. 11b2—A view of the FIG. 11b1 showing after the lifting plate 51 is pulled up to initiate the “chain reaction” to cause semi-circled semi-opened right and left sac airflow channel being further opened by the tightened sac wall descending edge.

FIG. 11c1—A cross sectional view through the air tube distal end 29, the lifting plate 51 is in a relaxed un-pulled position.

FIG. 11c2—A view of the FIG. 11c1 after the lifting plate 51 being pulled up. Consequently, the right and left edge of the lifting plate 51 are contacting the inner surface of the air sac wall and pushing right and left sac wall 81 toward the right and left side.

FIG. 11d—Cross sectional view through the air tube, showing the top tube airflow channel 30, the right tube airflow channel 32 and the left tube airflow channels 31, and a cross section of the first drainage tube 101 and second drainage tube 102.

FIG. 11e—Cross sectional view through of an alternative design of the air tube, showing how a semi-ellipse shaped air tube lumen can be divided by the drainage passage which includes the first drainage tube 101 and second drainage tube 102.

In similar fashion the air tube lumen can be divided as a top tube airflow channel, a right tube airflow channel and a left tube airflow channel.

FIG. 12—Multiple cross-sectional view lines of the device, the pulling unit not shown.

FIG. 12a—A cross sectional view through the sac distal pocket 99.

FIG. 12b1—A cross sectional view through the sac wall descending edge of the air sac. A cross section of the semi-circled semi-opened right and left sac airflow channel 78,79 are not yet being further opened before the lifting plate is pulled to cause the sac wall descending edge to be pulled and stretched. The sac airflow channel side walls 76 are pained darker line. The bottom fluid grooves 77 are shown in a cross section.

FIG. 12b2—A view of the FIG. 12b1 showing after the lifting plate 51 is pulled up to initiate the “chain reaction” to cause semi-circled semi-opened right and left sac airflow channel 78,79 being further opened by the tightened sac wall descending edge.

FIG. 12c1—A cross sectional view through the airflow distal edge 29, the lifting plate 51 in un-pulled position, The left tube airflow channel 31 and the right tube airflow channel 32 have already transitioned to the right and left sac airflow channel 78,79. The cross section of the bottom fluid grooves the distal drainage tube 109 are shown. The sac airflow channel side walls 76 is painted darker. The sac airflow channel middle wall 73, the sac airflow channel bottom wall 75 and the bottom fluid groove 77 are also shown. The lifting plate 51 is in a relaxed un-pulled position. The distal airflow bottom wall 29a, the distal airflow side wall 29b and the central groove 29c are shown.

FIG. 12c2—A view of the FIG. 12c1 after the lifting plate 51 being pulled up. Consequently, the right and left edge of the lifting plate 51 are contacting the inner surface of the sac wall 81 and pushing right and left sac wall 81 further toward the right and left side. The right and left sac airflow channel 78,79 are further opened. The spilt space 18b is labeled.

FIG. 12d—Cross sectional view through the air tube distal segment 15, showing outside shape of air tube as square like, the top tube airflow channel 30, the right tube airflow channel 32 and the left tube airflow channels 31, and a cross section the drainage passage 100 which is combination of the first drainage tube 101 and second drainage tube 102.

FIG. 12e—Cross sectional view through the air tube proximal segment 13 distal to the y point 18, showing how a circle shaped air tube lumen can be divided by the drainage passage. As similar fashion as the air tube distal segment 15, the air tube lumen can be divided as a top tube airflow channel 30, a right tube airflow channel 32 and a left tube airflow channel 31.

FIG. 13—Posterior view of the device, Show the right and left bottom fluid grooves 77, the distal drainage tube 109, alternative design of having two the band anchors 65a and having two the hooks 28, one on the back of the air tube and another on the back of the drainage passage 100.

FIG. 14a, left side view of a simplified drawing of alternative design, focus on showing the alternative design of the distal airflow tube 38 is painted as striped area. The distal airflow tube 38 is pre-made as an up-curved. The device is in process of an insertion and the air tube distal segment has been bent by an operator's hand, the split space 18b which is space between the distal airflow bottom wall 29a and the sac central protruding surface 39 become very small. The divergent plate lower edge 28c is suspension in the air. The divergent plate stopper 28d is on the air tube bottom wall in this drawing, but it can be configured on the air tube side walls. The left band anchor 65a the left pulling band 66 and the un-pulled lifting plate 51 are shown.

FIG. 14b—left side view of 14a, after the device has been inserted into throat and the lifting plate 51 has been pulled up by the pulling bands 66. The bent air tube distal segment has been relaxed in some degree, the distal airflow bottom wall 29a is away from the sac central protruding surface 39. The split angle or space 18b is greater. Increased the airflow pressure inside the right and left tube airflow channel during the intermittent positive pressure ventilation has pushed the divergent plate 28a to swing distally, the divergent plate lower edge 28c has reach the divergent plate stopper 28d and been stopped by the divergent plate stopper 28d.

FIG. 14c Top view simplified drawing to show the distal airflow bottom wall 29a, the central groove 29c, and the right and left divergent plates 28a, the divergent plate lower edge 28c and the divergent plate upper edge 28b which is painted as double lines. The rest of related structures using dotted lines. Please note that width of the right and left divergent plate 28a are narrower than the right and left tube airflow channel 32, 31.

FIG. 15a—showing the third embodiment configuration, no any type of drainage tubes are configured. The lifting plate 51 is pulled up and the air sac 119 is wide open. The sac bottom wall is optionally configured to have the sac central thickening zone 39a on the sac bottom wall therefore the sac bottom wall is divided into the right and left sac airflow channel 118a, 118b. The inward strip 86, sac wall descending edge 82, sac distal pocket cover 121, the air tube distal opening 117, the tube groove 118c and the sac distal plug 122 are shown. The pulling unit is same as in the first and second embodiment.

FIG. 15b. Simplified drawing of a distal portion of the third embodiment focus on showing the distal airflow segment 118 and air holes 116. The air tube distal opening 117 becomes smaller in cross section area than its beginning at where the distal airflow segment 118 transitions into the air tube distal segment 15.

### LEGEND LABELS

First Embodiment 10—device 12—air tube lumen 13—air tube proximal segment 15—air tube distal segment 16—right or left air tube side wall 17—air tube bottom wall 18—Y point 19—air tube anterior or top wall 20—air tube proximal opening 21—upward ramp central groove 22—upward ramp 23—downward ramp 25—indentation line 28—hook or hooks 29—air tube distal opening or air tube distal edge 30—top tube airflow channel 31—left tube airflow channel 32—right tube airflow channel 33—drainage passage shared middle wall 35—drainage passage top wall 36—right or left drainage passage side wall 37—drainage passage bottom wall 39—sac central protruding surface

51—lifting plate 52—right or left plate side edge 53—plate distal edge 55—right or left plate sealing petal 56—right or left corner sealing fold 57—right or left plate sealing bump 58—right and left epiglottis elevation plates 59—central epiglottis elevation plate 90—sac sealing belt 83—right or left sac wall holding zone angle (55—right or left plate sealing petal, 56—right or left corner sealing fold , 57—right or left plate sealing bump, 90—sac sealing belt, 83—right or left sac wall holding zone angle, all together collectively called enhanced sealing means)

60—pulling unit 61—band proximal junction 62—band hole 63—band tail 64—band bridge 65—band sliding pad 66—right or left pulling band 68—right or left band distal end 69—band distal converged

70—sac top opening 71—sac vertical attachment edge 72—sac horizontal attachment edge (sac vertical attachment edge 71 and sac horizontal attachment edge 72 together collectively called sac proximal attachment) 73—sac airflow middle wall 75—sac airflow channel bottom wall 76—sac airflow channel side wall 77—right or left bottom fluid groove 78—right sac airflow channel 79—left sac airflow channel

80—air sac 81—right or left sac wall 82—right or left sac wall descending edge 83—sac wall holding zone angle 85—sac wall holding zone 86—right or left inward strip 87—sac distal edge 88—sac wall proximal edge 89—sac distal pocket cover

90—sac sealing belt 91—lower horizontal edge 92—upper horizontal edge 93—umbrella distal edge 95—bar lifting assembly 96—right or left side fold 97—central fold 98—right or left lifting bar 99—sac distal pocket

100—drainage passage 101—first drainage tube 102—second drainage tube 104—second drainage tube proximal opening 108—second drainage tube distal opening 103—first drainage tube proximal opening 105—distal drainage tube distal opening 106—right or left distal drainage tube side wall 107—distal drainage tube bottom wall 109—distal drainage tube 111—central drainage tube 114—central drainage tube proximal opening 113—central drainage tube distal opening 112—fluid entrance 115—fluid hood

### Second Embodiment

15a—distal airflow channel 18b—split angle or split space 18c—spit point 28a—right or left divergent plate 28b—divergent plate upper edge 28c—divergent plate lower edge 28d—divergent plate stopper 29a—distal airflow bottom wall 29b—right or left distal airflow side wall 29c—central groove 29d—incomplete top wall 38—distal airflow tube 65a—right or left band anchor 66a—right or left pulling stick 99a-sac lifting plate

### Third Embodiment

39a—sac central thickening zone 116—air holes 117—air tube distal opening or air tube distal edge 118—distal airflow segment 118a—right sac airflow channel 118b—left sac airflow channel 118c—tube groove 119—air sac 119a—air tube proximal segment 119b—air tube distal segment 120—air tube lumen 121—sac distal pocket cover 122—sac distal plug 123—sac distal pocket

### DETAIL DESCRIPTION OF THE INVENTION

### First Embodiment

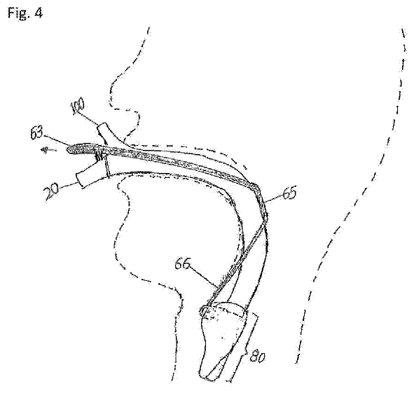

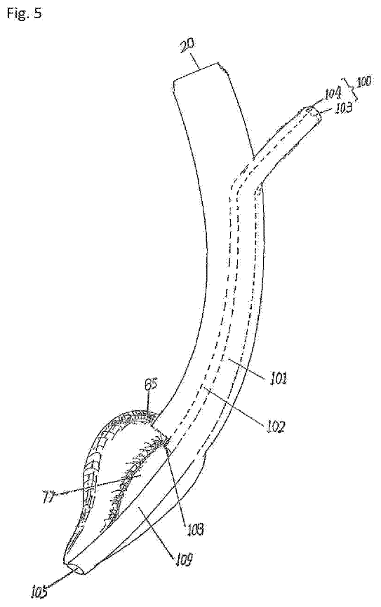

The first embodiment comprises an air tube, an air sac, a tongue lifting means, and pulling unit 60 with a drainage system, FIG. 1-11, 13.

### Air Tube:

FIG. 1, 2, 3, 4, 5, 6a, 6c, 7a, 7b, 8c, 9a, 9b, 9c, 9d, 10a, 10b, 10c, 11, 11a, 11b1, 11b2, 11c1, 11c2, 11d, 11e, 13, the air tube is a hollow tube includes an air tub proximal segment 13 and an air tube distal segment 15, and is opened at the air tube proximal opening 20 and at air tube distal opening 29, also interchangeably called air tube distal end or edge 29. The air tube 11 can be subjectively divided into an air tube proximal segment 13 and an air tube distal segment 15. Preferably the air tube distal segment 15 is curved anteriorly. The air tube proximal opening 20 is configured to be connected with any currently used respiratory equipment.

A cross section of outside shape of the air tube can be configured in different geometric shapes. The air tube lumen cross section can be in a circle, semicircle, ellipse, semi-ellipse, oval, squares, rectangles, trapeziums, or a combination. Preferably, the air tube distal segment 15 in the present invention is configured as a rectangle-like shape, with rounded corners where it would contact soft tissues of pharynx, FIG. 11d.

In first embodiment of the present invention the air tube outside shape is different from its inside shape. The air tube outside shape in cross section can be configured as horizontal rectangle and is surrounded by an air tube bottom wall also called posterior wall 17, an air tube top wall also called anterior wall 19, and a right and left air tube side walls 16.

Shape of the air tube lumen varies along the air tube length. A drainage tube, called drainage passage 100, has smaller cross section area than air tube and is configured inside the air tube lumen 12 and couple to the air tube middle bottom wall longitudinally fora length starting from the air tube distal end 29 toward the air tube proximal opening 20. About three to eight centimeters from the air tube proximal opening 20, the drainage passage separates from the air tube and bends posteriorly away from the air tube bottom wall 17, or the air tube bends anteriorly away from the drainage passage 100, thereby forming a “Y” shaped separating point, called a Y point 18. Or in other words, at the Y point 18 the drainage passage 100 “invade” longitudinally into the air tube lumen and follows the air tube in same direction passing beneath the air tube distal opening 29 and share the middle strip of the air tube bottom of air tube bottom wall 17 in side of the air tube lumen. From they point to the air tube proximal opening 20, the shape of the air tube inside and outside lumen can converge and gradually becomes circular to be connected with industrial standard respiratory equipment.

The cross section of the drainage passage 100 can be configured as different geometric shapes same as a cross section of the air tube, and preferably is a rectangle or square shape. The drainage passage 100 can be configured differently in different embodiments but they all do not communicate with air tube lumen. In the first embodiment, the drainage tube 100 includes a first drainage tube 101 and a second drainage tube 102. The both tubes are hollow fluid drainage tubes, share a common wall 33 and do not communicate with each other.

Therefore from the Y point 18 to the air tube distal opening 29, the actually cross section area of the air tube lumen has is reduced, and the drainage passage is divided or reshaped the air tube lumen to an inverted “U” shape with a top bar and two legs on right and left side in a cross section. In other words, in a cross section view, outside shape of the air tube looks like a rectangle, but inside shape of the air tube lumen looks like an inverted “U” shape. The top bar or upper part of the air tube lumen is called top tuber airflow channel 30, right and left legs on right and left side of the drainage passage 100 are called right tube airflow channel 32 and left tube airflow channel 31 respectively. Please note that there is no completely physical dividing line among the three airflow channels and they are in fluid communication. In one configuration, all three airflow channel stop just at the conjunction between the air tube and the air sac, or the right and left tube airflow channel 32,31 can extend further distally. The end of the top tube airflow channel is called the air tube distal opening 29. Therefore the cross section of the air tube lumen prior to the Y point 18 has very different shape than at the air tube distal opening 29. The air tube lumen in cross section view at the air tube distal opening 29 is a sum of cross sections of the top tube airflow channel 30, the right tube airflow channel 32 and the left tube airflow channel 31. The air tube lumen in cross section view at the air tube proximal opening 20 is circular-like shaped FIG. 11e.

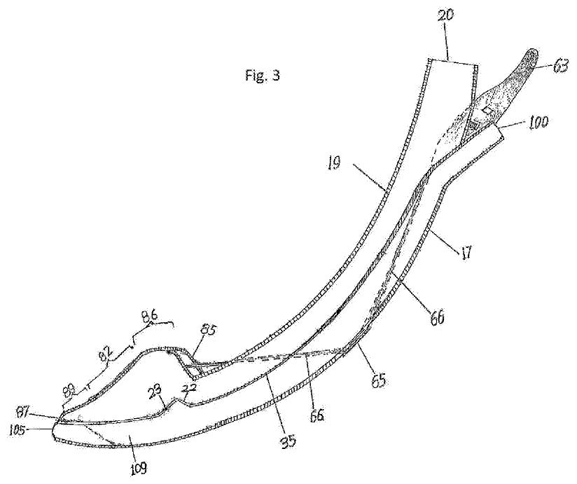

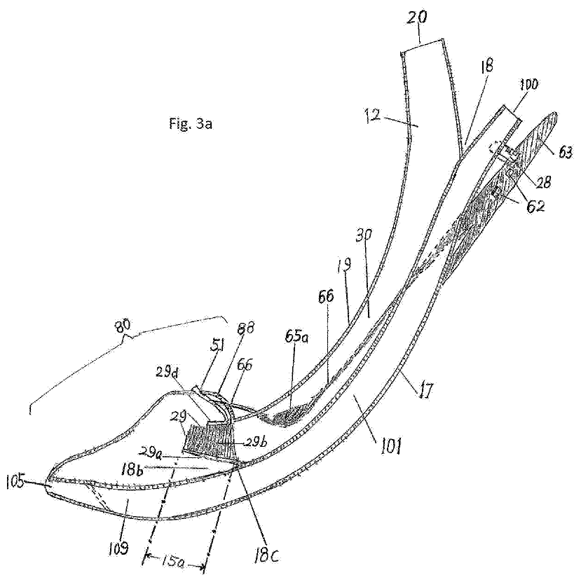

AS shown in the FIG. 3, 6b, 11c1, 11c2, 11d, the drainage passage top wall 35 will be at the bottom of a tube top airflow channel 30. The right tube airflow channel 32 is surrounded incompletely by the air tube right side wall 16, the right drainage passage side wall 36 and a right strip of the air tube bottom wall 17. A left tube airflow channel 31 is incompletely surrounded by the left drainage passage side wall 36, the air tube side wall 16 and a left strip of the air tube bottom wall 17.

When an airflow enters into the air tube proximal end 20, after passing the Y point 18, the airflow will be divided and flow into these three channels toward the air tube distal end opening 29. The right tube airflow channel 32 is configured to continue with the right sac airflow channel 78, and the left tube airflow channel 31 is configured to continue with left sac airflow channel 79. They will be discussed more later.

The air tube distal segment 15 is preferably flexible and resilient so that the tubes can be flexed to follow the curvature of the back of the person's or animal's tongue without requiring excessive force during insertion. In a preferred embodiment, the air tube distal segment 15 can have a preformed upward curve. And this preformed upward curve can be curved more under a force. The air tube distal segment 15 can be constructed by same material as the air tube proximal segment 13 or by different materials. Preferably the air tube is formed from a type of material that is preferably transparent and can provide operator visualization through the walls of the air tube.

The air tube is of sufficient length to extend from the laryngeal opening to beyond the lips of a human or animal. Preferably the air tube can be configured with a bite guard on the air tube proximal segment just distal from the Y point. The bite guard is sufficiently rigid to prevent a patient or an animal's teeth biting down or cutting off or excessively restricting lumens of the air tube.

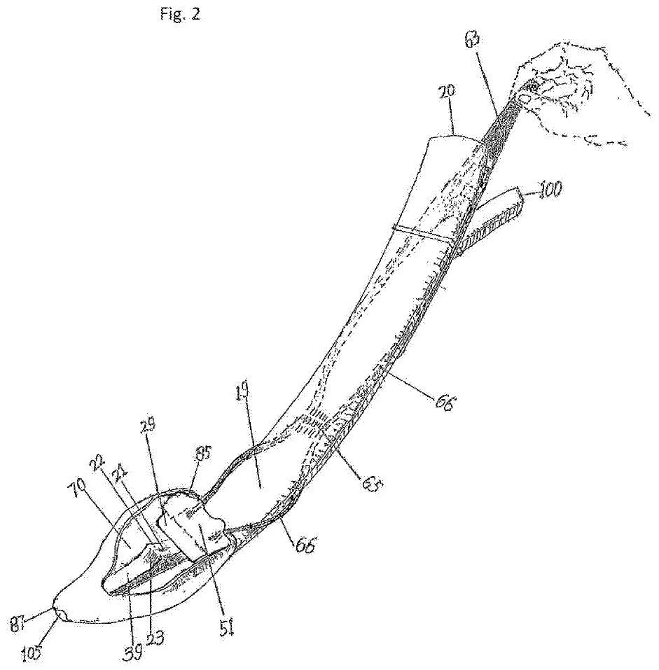

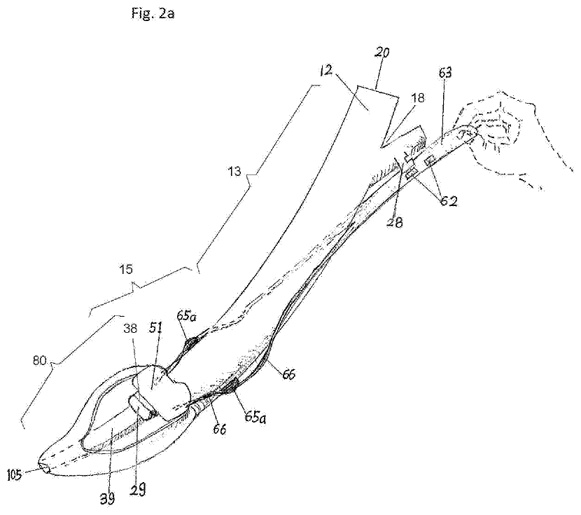

After passing the air tube distal opening 29, the drainage passage top wall bends upward forming a short upward segment, called an upward ramp 22, then bends down to it original level, called a downward ramp 23 FIG. 2, 3, 7a. Alternatively, the upward ramp and the downward ramp can be configured as a convex arch without an angle. In the middle of the upward ramp 22, preferably a groove like depression is configured longitudinally, called an upward ramp groove 21. The upward ramp groove 21 can provide guidance for passing a stylet, or a suction catheter into laryngeal inlet. If intubation is needed, an operator can manipulate a stylet tip to follow the upward ramp groove 21 into a laryngeal inlet. Once the stylet is inside of the tracheal, the operator can follow commonly used intubation steps to finish intubation. In an alternative design the upward ramp groove can be omitted, the upward ramp can still help to guide the tip of a stylet into the vocal cords opening.

The upward ramp 22 is a structure inside of the air sac and can guide the tube top airflow toward the laryngeal inlet once the airflow exits the air tube distal end 29 and therefore creates a direct airflow. The downward ramp 23 is configured to not obstruct the airflow from the sac distal pocket 99. After the drainage passage 100 pass beneath the air tube distal end 29, it continue with the distal drainage tube 109. The distal drainage tube terminates at the air sac distal edge 87 FIG. 3, 6a, 8c.

The directed air flow can create a more laminar air flow and reduces turbulent flow, therefore reduces airway resistance and work of the breathing which can play an important role during ventilation. In case of positive pressure ventilation, the direct air flow requires less inspiratory pressure to achieve same tidal volume. Subsequently possibility of air leakage and the air being forced into the stomach are also reduced. Therefore fewer complications and more effective positive pressure ventilation are achieved. This is one of important advantages of the present invention over currently used supraglottic airway devices.

### Air Sac

The air sac, also called a sac, is a structure that “extended” distally beyond air tube distal opening 29. The air sac 80 is configured like a “shoe” with “cutting off” posterior portion of the “shoe”. Space inside of the “shoe” is called air sac chamber. Top opening of the “shoe” is called sac top opening 70. The “toe box” of the shoe is called sac distal pocket 99. The most front end of the “toe box” is called air sac distal edge 87. The proximal end of the air sac is coupled to the air tube distal segment 15, FIG. 1, 2, 3, 6a, 8c, 9a, 9c, 9d. Bottom of the “shoe” is called sac bottom wall.

The surface of the sac bottom wall is not even. On the surface of the air sac bottom wall, a central longitudinal protruding structure protrudes into the air sac chamber starting from the air tube distal opening 29, and extending to the air sac distal edge 87, called sac central protruding surface 39 which include the upward ramp 22 and the downward ramp 23. The sac central protruding surface 39 is “sandwiched” by two depressed semi-circled like structures along its right and left side longitudinally, respectively called a right sac airflow channel 78 on its right side and a left sac airflow channel 79 on its left side. The shape of the sac central protruding surface 39 is configured to accommodate the distal drainage tube 109. Preferably the sac central protruding surface 39 of the sac bottom wall shares the top wall of the distal drainage tube 109. Or alternatively the sac central protruding surface 39 has its own separated layer above the top wall of the distal drainage tube 109.

In a cross section view FIG. 11b1, 11b2, 11c1, 11c2, the sac airflow channel is a semi-circled or semi-ellipse like semi-open structure and has a convex side wall laterally, wall called a sac airflow channel side wall 76, and has a curved bottom wall, called sac airflow channel bottom wall 75, and has a sac airflow channel middle wall 73 which bends obliquely form the sac airflow channel bottom wall 75 to the sac central protruding surface 39.

Longitudinally, the sac airflow channel side wall 76 extends up vertically further to form a trapezoid shaped plate on top of the sac airflow channel side wall 76, called a sac wall 81. Or in other words the right or left sac wall 81 fuses or transitions into the right or left sac airflow channel side wall 76 smoothly. The right or the left the sac walls 81 has a concave shaped inner surface toward each other. The right and left sac airflow channel 78,79 and the right and the left sac wall 81 are preferably molded together during manufacture.

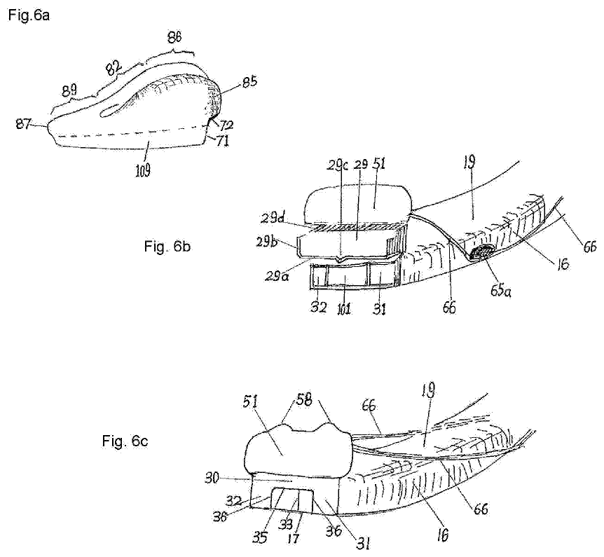

The right and left most proximal edge of the sac wall are called sac wall proximal edge 88. A proximal portion of right or left sac wall 81 is extend proximally 0.5-3 centimeter above the air tube top wall and beyond the air tube distal end 29, this portion of the right and left of the sac wall 81, either on right or left side called sac wall holding zone 85. The right and left sac wall holding zones 85 are “standing” on the air tube top wall 19 vertically or almost vertically. The right and left sac wall holding zones are bent toward the middle and are vertically perpendicularly or near perpendicularly attaching to the air tube top wall. Therefore 0.5-3 centimeter length of the bottom line of the sac wall holding zone 85 is called sac horizontal attachment edge 72. The right and left the sac horizontal attachment edge 72 are coupled to top wall of the air tube distal segment 15 starting at right and left of the corner of the air tube distal edge 29 toward to the middle. Accordingly the right and left of the sac wall holding zone 85 start to bend toward to each other at right and left side converged corner of the air tube top wall 19 and the right and left air tube side wall 16 at the air tube distal edge 29. After the right and left sac wall holding zone 85 bend toward each other, the right and left sac wall proximal edge 88 are getting closer to each other but do not touch each other in the middle. The right and left sac wall proximal edge 88 are perpendicular or almost perpendicular to the air tube top wall 19 and are also the proximal edges of the right and left sac wall hold zone 85, FIG. 6a, 9a. There is a space between the right and left sac wall proximal edge 88. The right and left pulling band 66 pass through the space between the right and left side of the sac wall proximal edges 88 and touch right and left sac wall proximal edges 88 respectively during the passing. When the pulling band 66 are pulled in tension, the band distal segment will push the right and left sac wall proximal edges 88 further apart. The most proximal end of the sac airflow channel side walls 76 of the sac airflow channel 78,79 is firmly attaching to the right or left air tube side wall 16 respectively, called sac vertical attachment edge 71, FIG. 6a, 8a, 9a, 9b. The sac vertical attachment edge 71 and the sac horizontal attachment edge 72 together function as an attachment tool to connect the air sac to the air tube distal segment 15. The right sacs vertical attachment edge 71 and the right sac horizontal attachment edge 72 together constitute a right sac proximal attachment. The left sacs vertical attachment edge 71 and the left sac horizontal attachment edges 72 together constitute a left sac proximal attachment. They are collectively called sac proximal attachment.

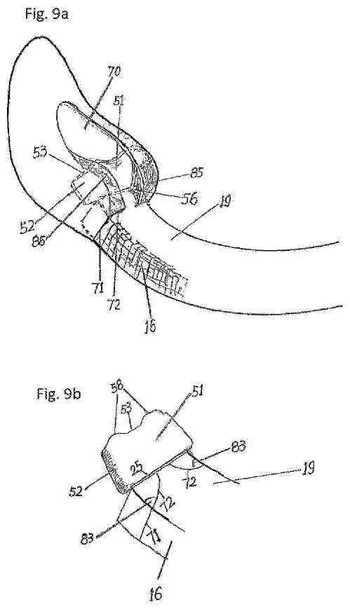

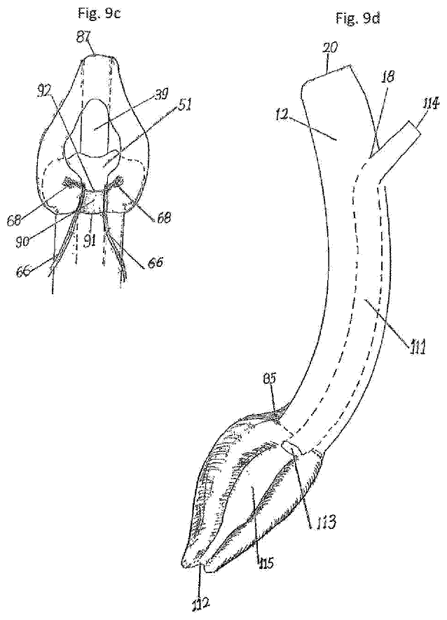

In one embodiment the right or left the sac vertical attachment edge 71 continue with right and left air tube side wall at the air tube distal edge 29, so there are no physical attachment lines visible. Alternatively the air sac vertical attachment edge 71 can be configured to extend beyond the air tube distal end 29 and firmly attach the air tube side wall 16 of the air tube distal segment 15, FIG. 9a, 9b. The attachment line of sac vertical attachment edge 71 preferably is within 3 centimeter distance from air tube distal edge 29. Accordingly the sac horizontal attachment edge 72 will attach further proximally from the air tube distal end 29 on the air tube top wall 19. In this alternative design, FIG. 9b, the right or left the sac horizontal attachment edge 72 attaches the air tube top wall 19 first and then turns backward to the indention line 25 at where a lifting plate 51 couples with the air tube distal end 29. The right or left sac horizontal attachment edge 72 will form an angle with right or left side edge line of the air tube top wall 19, called the sac wall holding zone angle 83, FIG. 9b. Therefore the right and left sac holding zone 85 will perpendicularly couple to the air tube top wall 19 and bending obliquely facing to each other. When the lifting plate 51 is pulled up and upper surface of the lifting plate will contact with right and left of the sac wall proximal edges 88 to form a seal to prevent the air leaking out from inside the air sac. The right and left sac wall holding zone angle 83 are one of enhanced sealing means or mechanism.

The function of the right and left sac wall holding zone 85 are that when a tongue lifting means is pulled up by the pulling unit 60 the right and left the sac wall holding zone 85 will contact and hold the lifting means to prevent the lifting plate 51 from being pulled too far proximally. Therefore the right and left of the sac wall holding zone 85 create a resistance to the lifting plate 51 when the tongue lifting means is fully pulled up. At same time the pulled the tongue lifting means will form a seal with the sac wall holding zone 85 to prevent air leakage. And at same time the sac wall holding zone 85 will be pushed by the tongue lifting plate means toward proximal direction.

The right and left sac airflow bottom walls 75 of the right and left the sac airflow channel 78, 79 are smooth continuation and transition to the right tube airflow channel 32 and left tube airflow channel 31 respectively. The right and left sac airflow channel 78, 79 are curving outward away from air tube distal opening 29 and then curving toward the middle to merge together. And the right and left the sac airflow channel bottom walls 75 turn up to fuse in the middle on the top of the distal portion of the distal drainage tube 109. Preferably the right and left the sac airflow channel 78,79 have same cross section area and are shaped symmetrically. The semi-circle shaped right sac airflow channel 78 and the left sac airflow channel 79 are smoothly changed to the square-like shaped right tube airflow channel 32 and the left tube airflow channel 31 respectively. The smooth changes in physical structure would make the airflow from the right or left tube airflow channel smoothly flow into right and left sac airflow channel 78,79 without meaningfully increasing the airflow resistance. The air or gases inside of right and left sac airflow channel 78,79 are freely communicating with the air sac chamber.

The right and left distal portion of the sac airflow channel 78,79 bend up and merge in the middle to form a pocket like structure with an opening toward the air sac chamber, called the sac distal pocket 99. Top wall of the sac distal pocket 99 is preferably configured as a thinner layer and extends more proximally, called a sac distal pocket cover 89. The sac distal pocket cover 89 is preferably thin plastic or silicone like material with more flexibility and compressible. The sac distal pocket cover 89 can be compressed down during the device insertion and can be pushed up by airflow inside of the sac distal pocket 99 during inspiration phase.

The sac distal pocket 99 is positioned on top of the distal portion of the central longitudinal protruding surface 39 or the distal drainage tube 109. The distal side of the sac distal pocket 99 is closed. The airflow cannot flow out the distal end of the sac distal pocket 99. The airflow can only flow out from beneath of the sac distal pocket cover 98 toward the air sac chamber. Therefore airflow inside the sac distal pocket 99 can cumulate and then push up the easily flexed sac distal pocket cover 89 therefore to make the sac distal pocket cover 89 forming a tighter seal with the posterior laryngeal wall during the inspiratory phase of the respiratory circle, especially in intermittent positive pressure ventilation. The sac distal pocket cover 89 is the top wall and an integrated part of the sac distal pocket 99, but preferably it can be manufactured as a thinner layer therefore to have more flexibility and expandability than rest of the sac distal pocket.

Top edges of right and left sac wall 81 are bent inward toward each other but do not touch each other, and form a right and a left smooth bending narrow strip longitudinally on top of the right and left sac wall 81, called a right or a left inward strip 86. Longitudinally, when the right and the left sac wall's inward strip 86 are going distally and descend diagonally down to form right and left distal edges of the sac wall 81, called a right or a left sac wall descending edge 82. The right and left sac wall descending edge 82 are smoothly continuing and transition to the sac distal pocket cover 89.

The right and left inward strip 86 are obliquely positioned toward each other in a relaxed state before being pushed up by a tongue lifting means, such as the lifting plate 51. And their oblique positions will be turned upward when the right and left inward strip 86 are pushed up or “turned up” by the distal end of the rising lifting plate 51 therefore to open the sac top opening 70 further.

Since the right and left sac wall inward strips 86 are structurally continuing with the right and left sac wall holding zone 85 when the sac wall holding zone 85 are pushed proximally by the lifted lifting plate 51, the sac wall holding zone 85 will pull the inward strip 86. And since the inward strip 86 is continuing with right and left sac wall descending edges 82, the sac wall descending edges 82 are continuing with the sac distal pocket cover 89. The pulled inward strip 86 will pull the right and left sac wall descending edges 82, the right and left sac wall descending edges 82 will pull the sac distal pocket cover 89. When the sac distal pocket cover 89 is pulled tight and intense, it will form a tighter seal with posterior laryngeal wall. At same time, the tensed the right and left sac wall descending edges 82 will keep the right and left sac airflow channel 78,79 open and prevent it being compressed by surrounding pharyngeal tissues. All these reactions together form like a “chain reactions” than will keep the right and the left sac airflow channel 78, 79 and the sac distal pocket 99 open, and form the seals at differently regions around the laryngeal inlet, and prepare the right and left sac airflow channel 78,79 and the sac distal pocket 99 to be expanded by the incoming airflow.

The right and left sac wall descending edges 82 are significantly vertical or perpendicular in relation to right and left inward strip 86 and the sac distal pocket cover 89. After the device is inserted into a patient throat, the sac distal pocket cover 89 is to be under the posterior wall of the larynx, the right and the left sac wall descending edges 82 are to contact right and left side structures of the laryngeal inlet and create a resistance which can be felt by an operator' hand and therefore giving a stopping signal to stop insertion. This is another function of the right and left sac wall descending edges

The airflow in the tube top airflow channel 30 will flow toward the vocal cords opening. Airflow in the right and the left tube airflow channel will flow into the right and left the sac airflow channel 78,79 of the air sac 80 respectively. Then the airflow inside of the right and left sac airflow channel 78,79 will continue to flow distally into the sac distal pocket 99 to “inflate” the sac distal pocket 99. Finally the airflow flow out the sac distal pocket 99 from the beneath of the sac distal pocket cover 89 into the air sac chamber to participate in the air exchange. This channeled airflow will play important role to push the air sac to form a further conformal seal with pharyngeal walls and tissues around the laryngeal inlet during inspiration phase of a respiration cycle. And the pushing force is the inspiratory pressure dependent and is proportion to the inspiratory pressure. The pushing force will subside during the expiratory phase of a respiratory circle.

This channeled airflow can also dissipate the excessive pressure to make a further seal if an excessive pressure applied accidently by an inexperienced operator.

The sac distal pocket cover 89 is a thin plastic or silicone or other compatible material film on top of the sac distal pocket 99. Preferably it is made same material as the sac distal pocket 99 but manufactured thinner. The sac distal pocket cover 89 will be pushed up by inspired airflow inside of the sac distal pocket 99 especially during positive pressure ventilation to form a seal with posterior wall of the larynx, and also help to form a seal with upper esophagus opening. The seal, provided by sac distal pocket, can prevent the regurgitated esophageal fluid to ingress into the air sac, and at same time can prevent the ventilating gases from ingressing into the stomach or being pushed into stomach when spontaneous ventilation or positive pressure ventilation is applied.

The whole air sac is made of the materials with certain compressibility, elasticity and plasticity, such as plastics, polymers or other alike. The shape of the right and left sac airflow channel 78,79 and the sac distal pocket 99 can be compressed to a certain extent to accommodate local anatomy and during insertion of the device. The air sac would evenly distribute the sealing pressure on the surrounding tissues and not put too much pressure on a specific area of the pharynx or the larynx during forming the seal. The flexibility and elasticity of the air sac also makes the right and the left sac airflow channels and the sac distal pocket 99 able to be pulled or pushed for more opening and can be expandable when pressured airflow flow into the right and the left sac airflow channels and the sac distal pocket 99. All these features will accommodate each individual anatomy variations well and establish a seeming “self-sealing” mechanism.

Inside the space between the right and left sac wall 81 and the sac wall holding zone 85, a tongue lifting means and a portion of the pulling bands 66 are configured.

### Tongue Lifting Means

The tongue lifting means is a structure can be pulled up and consequently push up the tongue base. The tongue lifting means and pulling unit 60 are two different elements. They are connected or coupled to each other and working together.

The tongue lifting means can be configured as a lifting plate 51, or a bar lifting assembly 95. The both can be configured at the upper edge of the air tube distal end 29 and are lifted by the pulling unit 60. The both are used as a lifting means to push up the tongue base and can be collectively called the tongue lifting means.

The lifting plate 51 is a plate can be made of plastic, silicone, polymers and other like materials or even metal materials with some flexibility and elasticity features. The lifting plate 51 is preferable a distal extension of the air tube top wall 19 at the air tube distal end 29. Or alternatively an independent of plate couples with the top wall of air tube distal opening 29.

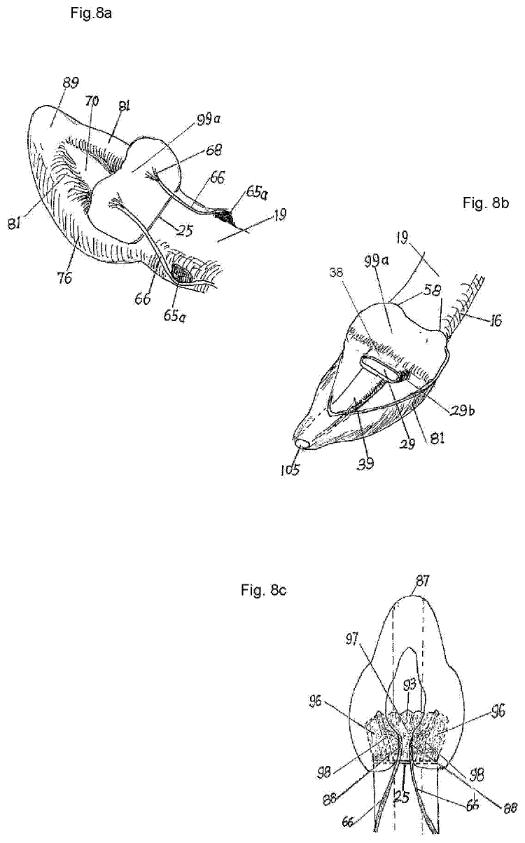

The lifting plate 51 can be different geometric shapes, such as: square, rectangle, trapezoid, round, elliptic or half elliptic, hexagon and so on, and can be flat, curved as concave or convex shapes at different regions of the lifting plate 51, angled or not angled and so on. In a preferred embodiment, the lifting plate 51 has trapezoidal shaped without sharp angles. The wider base side of the trapezoid of the lifting plate 51 is coupled to top edge of the air tube distal opening 29. The most distal edge of the lifting plate 51 in general is parallel to the top edge of the air tube distal edge 29, called plate distal edge 53, and is configured to be bent down when meeting a resistance from above. The right or left side of the lifting plate 51 is called right or left plate side edge 52. In relaxed un-pulled position, the lifting plate 51 is parallel or almost parallel with bottom wall of the air sac with its lower surface facing to the air sac bottom wall. One or two distal ends of the pulling bands 66 are coupled to upper surface of the lifting plate. When the pulling unit 60 is being pulled by an operator, the pulling bands 66 will pull the lifting plate 51, the lifting plate 51 will move up diagonally in which the plate distal edge 53 will move in a arch curved trajectory. Preferably the lifting plate 51 is manufactured as extension of the air tube top wall but is thinner than the air tube top wall 19. And the connection between the lifting plate 51 and the top edge of the air tube distal edge 29 is preferably configured to have a shallow indentation line, called the indentation line 25, FIG. 8c, 9b. The indentation line 25 is to make the connection between the lifting plate 51 and the top edge of the air tube distal edge 29 “weaker” therefore easy to be bent. The lifting plate 51 can use the indentation line 25 as a bending “joint” to form a dull angle with the air tube top wall 19 at the air tube distal end 29 when the lifting plate 51 is pulled up diagonally. The indentation line 25 is a part of the lifting plate 51.

The right and left plate side edges 52 are also configured to bend down slightly when the lifting plate is in a relaxed un-pulled position and can be bent more. When the lifting plate 51 is pulled up the right and left lifting plate side edges 52 will push the right and left sac wall to further outward, and at the same time the right and left lifting plate side edges 52 will be bent by the right and left sac side walls 81 further therefore to increase contact area with the concave inner surface of the right and left sac wall 81 to form seal which can prevent the airflow inside of the air sac chamber escaping from the proximal side of the air sac. The diagonal upward movement of the lifting plate 51 will make the lifting plate distal edge 53 contacts with the tongue base and be bent further and therefore forms a seal with the tongue base.

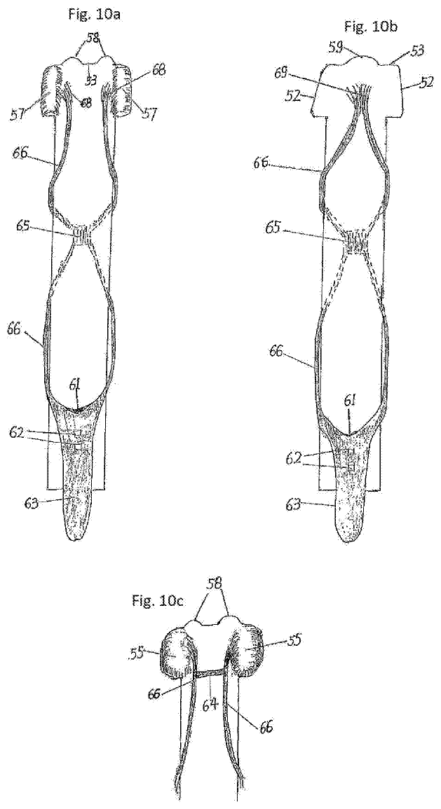

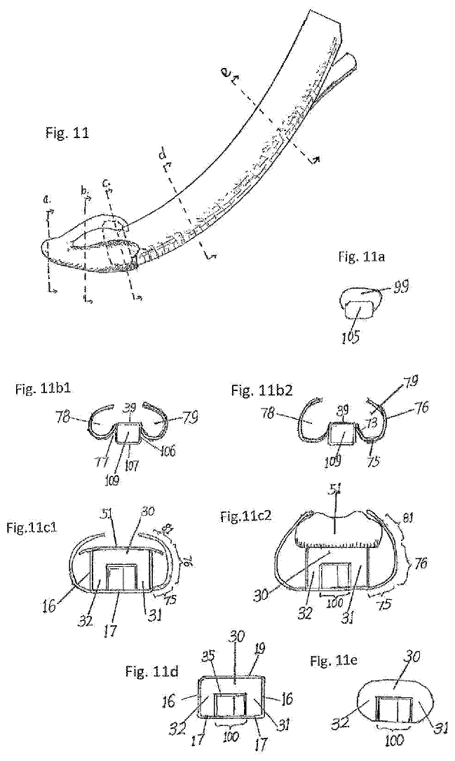

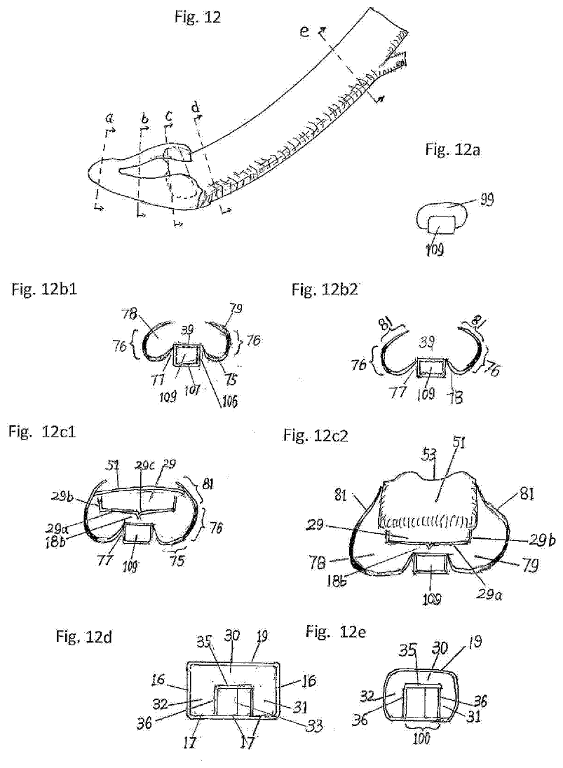

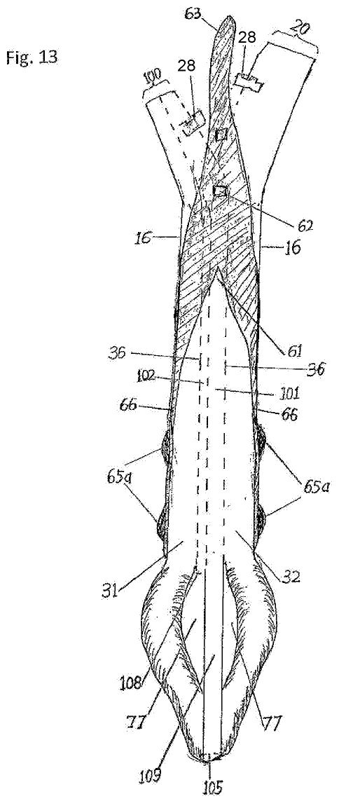

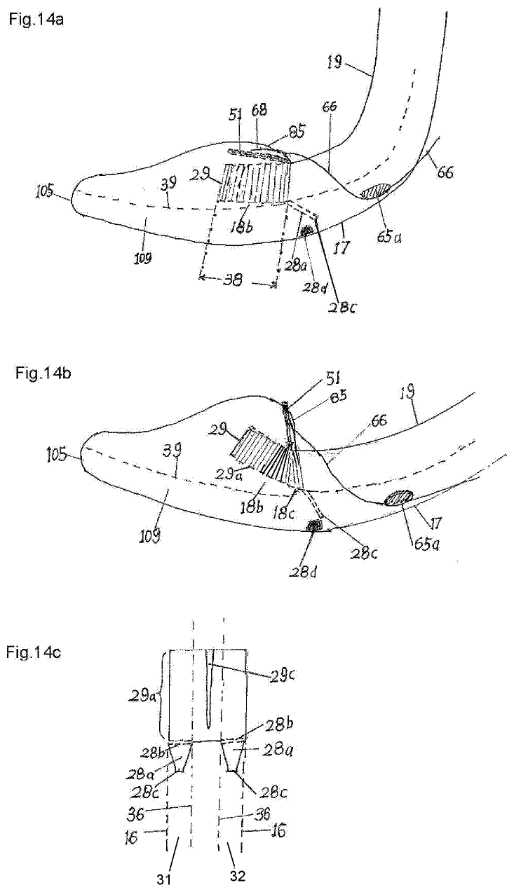

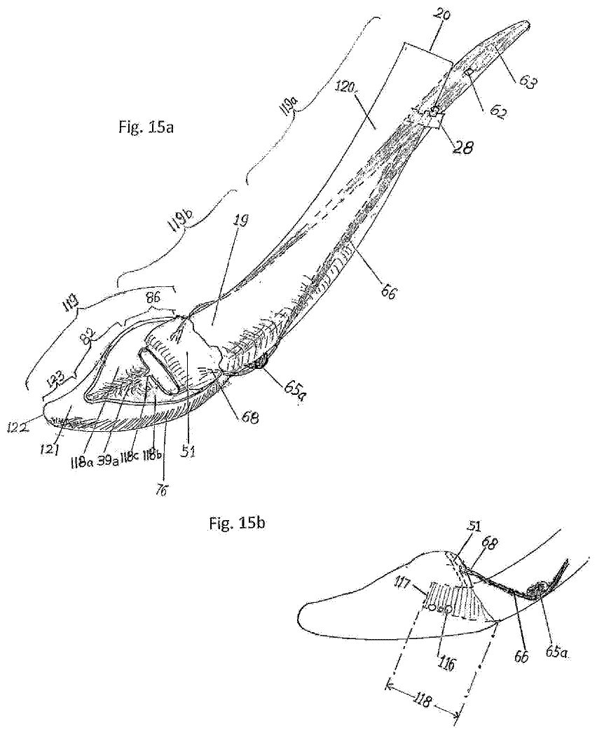

In a preferred embodiment, two round protrusions extending from the lifting plate distal edge 53 can be configured, called epiglottis elevation plates 58 FIG. 6c, 8b, 9b, 10a, 10c. Each of the two epiglottis elevation plates 58 is positioned on right and left side of the plate distal edge 53. Alternatively only one epiglottis lifting plate can be configured in the middle of the plate distal edge, called a central epiglottis elevation plate 59, FIG. 10b. Both types of the epiglottis elevation plates can be configured in many different shapes, preferably like round protrusions from the plate distal edge 53. They may or may not be on same horizontal plane as the lifting plate 51. The epiglottis elevation plates 58 and the central epiglottis elevation plate 59 are collectively called epiglottis elevation means. During the tongue lifting plate 51 being pulling up, the epiglottis elevation means will move up diagonally and contact the lower surface of the epiglottis either on the right and left side or center of the epiglottis, and then push up the epiglottis to open the laryngeal inlet. And they also are able to keep the epiglottis elevated while the lifting plate 51 is being kept at an elevated position. Therefore the laryngeal inlet will be kept open during ventilation. This mechanism will greatly facilitate laminar airflow. If the epiglottis elevation plates misses contact with the epiglottis, the epiglottis elevation plates will contact with the tongue base or nearby tissues. In ether situations, the epiglottis elevation plate will help increase the portion of the laminar airflow and effectiveness of intermittent positive pressure ventilation or spontaneous ventilation.

The epiglottis elevation plates are preferably made same materials as the lifting plate 51, and the lifting plate 51 s preferably is made same material as the top wall of the air tube distal segment. But they can be constructed with different materials. In an alternative embodiment, the epiglottis elevation plates 58 and the central epiglottis elevation plate 59 can be omitted.

In one embodiment, FIG. 8c, the tongue lifting means is configured like an “umbrella”. It is configured as two or more bars with three or more folds, called bar lifting assembly 95. The bars are belt shaped rod, called lifting bar 98 and have certain flexibility but less than the flexibility of the folds. When the bar lifting assembly 95 is in relaxed un-pulled state, the bar lifting assembly 95 has its proximal edge couples to the air tube distal end 29, and its distal end free in the air, called umbrella distal edge 93. And in this configuration, the right and left sac wall holding zone 85 are configured to extend more toward the middle of the air tube top wall 19 which make the right and left sac wall proximal edges 88 further close to each other. The bar lifting assembly 95 is horizontally or almost horizontally facing down the air sac bottom wall when it is in relaxed un-pulled position. Preferably two lifting bars are configured to separate further as the both bars extending distally further. Therefore the when lifting bars 98 are pulled up diagonally the each bar will contact inner surface of the sac wall 81 to push the sac wall 81 toward right and left side, and also the each bar will contact the sac wall holding zone 85 to push the sac wall holding zone 85 toward proximal direction. At same time the pulling band of the pulling unit 60 will be tightened and push the right and left sac wall proximal edges 88 to right and left side to further open the sac top opening 70. One fold is configured between the two lifting bars 98 and connects the two lifting bars 98, called a central fold 97. Another two folds connect the lateral edge of each lifting bar 98 to the inner surface of the right and left sac wall holding zone 85, called right or left side fold 96. The central fold 97 is shaped like a vertically wedge shaped without bottom triangle. The central fold 97 and the right and left fold 96 are thin film like and all of them can be easily folded expanded with more flexibility than the lifting bar 98. The umbrella distal edge 93 is flexible and bendable. when the bar lifting assembly 95 is pulled up diagonally, the umbrella distal edge 93 which includes the distal ends of the lifting bar 98 is to form a seal with the tongue base. The pulling unit 60 will have two distal ends and each of them will couple to a lifting bar 98. When the pulling unit 60 pulls the lifting bar 98, the bars will be diagonally moved up, the central folds 97 will be unfolded in the middle to the touch and to be easily bent to accommodate shape of the tongue base therefore to form a conformable seal with the tongue base. The right and left side fold 96 will accordingly unfold and prevent the air escape from the air sac chamber. The whole the bar lifting assembly 95 will function like previously described the lifting plate 51 to form seal with the sac wall holding zone 85 and prevent ventilation gases leaking out from the air sac chamber.

In one embodiment, a horizontal rectangle shaped plate, called sac sealing belt 90, is configured to connect lower portion of the right ad left sac wall proximal edges 88 which also are edges of the right and left sac wall holding zone 85, FIG. 9c. The sac belt 90 has upper horizontal edge 92 and lower horizontal edge 91. The right and left side edge of the sac sealing belt 90 is connect or smoothly fuses or transitions into the lower portion of the right and left the sac wall proximal edges 88 which are about ¼-¾ height of sac wall proximal edges 88. The lower horizontal edge 91 can be curved and straight line and is coupled to the air tube top wall 19 just proximal to the air tube distal opening 29 or the indentation line 25. The sac sealing belt 90 is to seal the space between the lifting plate 51 and right and left sac wall holding zone 85.

Alternatively, a portion of the lifting plate 51 along it's the right or left side edge area within 2 centimeter distance from the right or left plate side edge 52 respectively, can be configured like a round bulging-up column longitudinally, like a “speedy bump” on a driveway, called right or left plate sealing bump 57, FIG. 10a. When the lifting plate 51 is pulled by the pulling bands, the plate sealing bump 57 will move up diagonally with lifting plate and contact and form a seal with sac wall holding zone 85.

In another alternative design, portion of the lifting plate 51 on its the right and left side can be shaped like a half petal of a rose with its convex surface bulging toward upside during relaxed un-pulling horizontal position, FIG. 10c called plate sealing petal 55. When the lifting plate 51 is pulled by the pulling unit 60, the plate sealing petal 55 will move up diagonally too, and the right or left plate sealing petal 55 will contact and form a seal with the right and left sac wall 81 or the sac wall holding zone 85.

Further alternatively, FIG. 9a, a similar wedge-shaped thin film or alike with folding and unfolding capability like a “folding hand fan” has been configured to connect right and left side of the lifting plate 51 to the right and left the sac wall holding zone 85 the sac wall proximal edge 88, called right or left corner sealing fold 56. The both attachment lines are within 0.3-1.5 centimeter from the right or left side of the lifting plate side edge 52. The right or left corner sealing fold 56 is connecting the lower portion of sac wall holding zone 85 or lower portion of the sac wall proximal edge 88. The lower portion is about ½-¾ height of sac wall holding zone 85 when the lifting plate 51 is pulled up. The right and the left the corner sealing folds 56 also tightly attach to the air tube top wall 19 near air tube distal opening 29. The right and left corner sealing folds 56 are configured to seal the space between sac wall holding zone 85 and right and left lateral area of the lifting plate 51.

In above described five designs or alternative designs, the sac sealing belt 90, the plate sealing bump 57, the plate sealing petal 55, the corner sealing fold 56 and the sac wall holding zone angle 83, FIG. 9b, all of them are to enhance the sealing between the right and left sac wall holding zone 85 and the right and left side lateral are of the lifting plate 51, therefore they are collectively called enhanced sealing means. The enhanced sealing means serve as an important tool to prevent the gases leakage from the air sac chamber in the present invention and intends to seal the space between the tongue lifting means and the right and left sac wall holding zones 85. However the enhanced sealing means can be omitted.

### Pulling Unit

The pulling unit 60 FIG. 1, 2, 2a, 3, 3a, 4, 10a, 10b, 10c, 13, 15a are one or more bands and are designed to pull up the tongue lifting means, and consequently the tongue lifting means will push up the base of tongue. At the same time the pulling band 60 can pull up the tongue base independent of action of pulling the tongue lifting means. When the base of the tongue is raised, laryngeal inlet surrounding area will be open up more to allow the air flow more directly toward laryngeal inlet to generate a more laminar airflow. Because the pulling force is coming from an operator's hand, the operator can feel and adjust the puling force, such as in an obese patient who may need more force to push the tongue base to exposure the laryngeal inlet. Finally the right and left pulling bands 66 of the pulling unit 60 are positioned on right and left side of the tongue base. Therefore when they are tightened, the right and left pulling band 66 can hold and stabilize the tongue in an elevated position. With flexibility and elasticity, the right and left pulling bands 66 are able to accommodate the shape of the tongue base well for each individual.

There are many different ways to configure different pulling unit 60 to achieve a same goal. But basic concept of using an operator hand generated mechanical force to lifting the tongue base is same and an essential, and is within scope of the protection of this invention.

In one embodiment, the pulling unit 60 are two belt shaped flexible strips or bands which merge together at their proximal ends, in the middle and in the distal ends. Or in another way to describe, one long band is divided into two narrower strips a couple of times during its longitudinal course. The pulling unit 60 can be made by many different materials, such as plastic, silicone, other polymers, metals or even fabric, but plastic or silicone are preferred materials. Preferably the pulling unit 60 has certain elastic and flexible property which can limit the possibility of tissue damage.

The configuration of pulling band 60 FIG. 1, 2, 3, 10a, 10b, 10c, 13, includes a band tail 63, one or more band holes 62 on the band tail 63, a bands proximal junction 61, right and left pulling band 66, the band sliding pad 65 and one or two band distal ends. In one embodiment, the right and left pulling bands 66 converge at their distal end to form a single distal end to attach to the lifting plate 51, called the band distal converged end 69. In a preferred embodiment, the two distal ends of the right and left pulling bands 66 will be separately attach to right and left side of the lifting plate 51, called the right or left band distal end 68, FIG. 8b, 9b, 10a. Alternatively, at close to the end of right and left bands distal end, a band bridge 64 can be configured to connect the right and left pulling bands 66 before finally attach the right and left side of the lifting plate 51, FIG. 10c.

Proximal to the Y point 18, on posterior wall of the air tube or on the anterior wall of the drainage passage 100, a hook 28 is configured for holding the band holes 62 or the band proximal junction 61. Alternatively two hooks 28 can be configured on right and left air tube side wall 16 and one or more band holes 62 can be configured on the pulling band 66 of the pulling unit 60 respectively. Therefore the hooks 28 on the right or left air tube side walls 16 will hold the band holes 62 on the right or left pulling band 66 separately. The hook 28 or the hooks are to hold one of the band holes 62 or the band proximal junction 61. Therefore an operator can free a hand. And the distance from each band hole to its distal end is premeasured and predetermined. Therefore an operator can make an estimate for each patient and make an appropriate adjustment during operation. The more that the pulling distance is pulled, the more pulling force on the lifting plate 51 which will push up the tongue base further. And at same time the pulling force of the pulling unit 60 will also lift up the tongue base. Up lifting of the tongue base will open up more space around laryngeal inlet. Therefore the operator can adjust the pulling distance to change the tongue' position by putting different band holes or the bands proximal junction 61 on the hook 28 or the hooks in different clinical scenarios. Also respiratory parameters, such as tidal volume, airway resistance and so on can be used as references to achieve a balance between an individually optimized openness of the laryngeal inlet and unnecessary pressure on the tongue base or surrounding tissues.

The band sliding pad 65 is configured to slide against the air tube posterior wall to convey operator's pulling force to the right and left pulling bands 66 and to the lifting plate 51. And also the band sliding pad 65 is to keep the right and left pulling band 66 on the right and left side of the air tube to avoid being bitten by a patient's or animal's teeth. The band sliding pad 65 will slide against the air tube posterior wall and move proximally when the pulling tail 63 is pulled by an operator. However, alternatively the band sliding pad 65 can be omitted.

In a preferred embodiment, the right or left band distal end 68 separately attach to the right and left side of the lifting plate 51. The right and left pulling bands 66 are touching the sac wall proximal edges 88 when the pulling unit 60 are in a relaxed un-pulled state. When pulling band 66 is pulled the pulling band 66 will be tightened in tension and will push the right and left sac wall proximal edges 88 aside therefore to push the right and left sac wall holding zone 85 aside and to further open the sac top opening 70 and the tightened right and left pulling bands 66 will also directly lift the tongue in addition to pulling up the lifting plate 51. And the pull bands 66 on the right and left side can help to stabilize the tongue's position during the tongue being elevated.

In summary, an operator will have to pull the pulling unit 60 to change physical position of some parts of the device before the device can perform its functions. Before the lifting plate 51 is pulled, right and left inward strip 86 of the air sac are configured towards each other. But they are not in contact in the middle and there is gap between right and left inward turning. When tongue lifting plate 51 is pulled up diagonally by an operator, the right and left sac wall 81 are pushed outward by raising the lifting plate 51 to enlarge the sac top opening 70, FIG. 2, 2a, 11c1, 11c2, 12c1, 12c2. And the plate distal edge 53 will also push up the right and left inward strip 86 which will open the sac top opening 70 further. At same time raising the lifting plate 51 will push right and left the sac wall holding zone 85 toward proximal direction which consequently will stretch and pull the inward strip 86 in proximal direction. The right and the left inward strip 86 will stretch and tighten the right and the left sac wall descending edges 82 and then the right and left sac wall descending edges 82 will keep the right and left sac airflow channel open to be expanded further by incoming airflow and to form seal with right and left pharyngeal wall. This chain reaction also will tighten and encourage the sac distal pocket cover 89 to further contact posterior laryngeal wall to form a seal. This mechanical force generated sealing mechanism is a major mechanism of the present invention to form seal with surrounding structures and is to set a stage for function of a second sealing mechanism.

The second mechanism to make the air sac 80 to form further seal with the surrounding tissue is when the divided airflow from the air tube enter into right and left sac airflow channel 78,79 during inspiration phase of a respiratory cycle, especially during intermittent positive pressure ventilation, the pressurized airflow from the right and the tube airflow channel 32,31 into the right and left sac airflow channel 78,79 will push and expand the right and left sac airflow channel 78,79 and push right and left sac airflow channel side wall 76 further laterally to make further seal with right and left pharyngeal wall. Then the airflow flow into the sac distal pocket 99 to push up sac distal cover 89 to form a seal with posterior wall of the larynx. This is an airflow sealing mechanism which is synchronized with respiration circle. During inspiration, the airflow pressure is increasing inside the sac airflow channel 78,79 and the sac distal pocket 99 to form the seal. During expiration the airflow pressure is decreasing in side the sac airflow channel 78,79 and the sac distal pocket 99 to avoid constant pressure on surrounding tissues and to reduce chance of tissue damage. Finally the airflow flow out from the sac distal pocket 99 into the air sac chamber. This second sealing mechanism is that the inspiratory airflow pressure drive the divided airflow to follow specific route to create pressure to the sac airflow channel 78,79 and the sac distal pocket 99 to form seals.

The hand generated mechanical force by an operator to cause these deformation changes of the device as described above is essential prior to the device to perform its functions.

Drainage System At the present invention, drainage system FIG. 2, 2a, 3, 3a, 5, 6c 7a, 7b, 9d, 11d, 11a, 12a, 12d, 13 can be reconfigured few different ways in different embodiments. They can include the drainage passage 100, a distal drainage tube 109. Or the drainage system can be configured to have one or two drainage tubes inside the drainage passage 100 with or without the distal drainage tube 109. And the drainage passage 100 can be configured different ways. All these arrangements and re-arrangements are within protection scope of the present invention.

In the first embodiment, the drainage passage is configured to include a first drainage tube 101 and a second drainage tube 102. The first drainage tube 101 continues distally with the distal drainage tube 109 therefore the both constitute the major drainage system in the first embodiment. The second drainage tube 102 can be omitted.

The first drainage tube 101 and the second drainage tube 102 are both hollow tubes and have their own proximal opening and their own distal opening, called the first drainage tube proximal opening 103, second drainage tube proximal opening 104, and second drainage tube distal opening 108.

At the proximal end of the drainage passage 100, there are a second drainage tube proximal opening 104 and a first drainage tube proximal opening 103 to be connected to an outside suction separately. The drainage passage 100 has a top wall, called drainage passage top wall 35, right and left drainage passage side wall 36. And its bottom wall shares the middle portion of the air tube bottom wall longitudinally. The both travel together inside the air tube lumen and are positioned next to each other and are sharing a common middle wall 33, collectively called the drainage passage 100. At the present invention the second drainage tube 102 is on left side of the first drainage tube 101, but this position can be switched.

Cross sections of the first drainage tube lumen and the second drainage tube lumen can be formed in different geometric shapes and their variations, such as: square, rectangle, circle or semicircle and so on. In present invention, the both lumens are a rectangle or square alike. And preferably the lumen size of the second drainage tube is smaller than the first drainage tube 101. Alternatively the both lumen size can be same. The total cross section area of the both lumens is smaller than air tube lumen cross section area.

After passing beneath of the air tube distal opening 29, the first drainage tube 101 continues distally with the distal drainage tube 109. Or the first drainage tube 101 and the distal drainage tube 109 can be described as one drainage tube and just change the name to the distal drainage tube 109 after passing underneath the air tube distal opening 29. A distal opening of the distal drainage tube 109, called distal drainage tube distal opening 105, terminates at the air sac distal edge 87 under the sac distal pocket 99.

Function of the first drainage tube 101 is together with the distal drainage tube 109 to drain the fluid from the upper esophagus. Once the first drainage tube 101 passes the air tube distal end 29, the distal drainage tube 109 travels beneath the air sac bottom wall and protrudes into the air sac chamber longitudinally to form the sac central protruding surface 39 as previously described. In cross section the distal drainage tube 109, square or rectangle shaped the distal drainage tube 109 has a distal drainage tube bottom wall 107, a distal drainage tube right and left side wall 106 and a top wall. The top wall of the distal drainage tube 109 shares the longitudinal middle portion of the air sac bottom wall which is on the top of the central longitudinal protruding surface 39. When the air sac is in a horizontal position, the distal drainage tube right or left side wall 106 is vertically positioned and the sac airflow middle wall 73 is obliquely traveling from the sac central protruding surface 39 down to the sac airflow channel bottom wall 75. Therefore viewing form the bottom of the air sac, there are two longitudinal gaps along the right and left the distal drainage tube side wall 106, called the right or left bottom fluid groove 77. The right or left bottom fluid grooves 77 are formed by the right and the left distal drainage tube side walls 106 and the right and left sac airflow middle walls 73. The right or left bottom fluid groove 77 are like a triangle shaped hood in a cross section to let fluid or secretions on the posterior pharyngeal wall cumulated there and to be suctioned out by the second drainage tube 102. The rhythmic expansion of the right and left sac airflow channel 78,79 during respiratory circle can facilitate accumulation of the fluid or secretion under the right or left bottom fluid groove 77. The distal drainage tube bottom wall 107 may or may not reach same lowest level of the sac airflow channel bottom wall 75.

Alternatively the sac airflow middle wall 73 can share the vertically positioned the right and left distal drainage tube side walls 106 which will make the right and the left sac airflow channel 78,79 become square like in a cross section. Therefore in this alternative design there will be no the right and left bottom fluid groove 77.

The lumen of the first drainage tube 101 and the distal drainage tube 109 can be configured big enough to allow a fiber-optic-scope-probe or a currently used smaller oral-gastric tube passing through into a patient's stomach.

The second drainage tube 102 has its second drainage tube distal opening 104 passing beneath the air tube distal end 29 and terminates at the right or left bottom fluid groove 77. The second drainage tube 102 is to drain the fluid or secretion cumulated under the right or left bottom fluid groove 77 where the fluid cumulate between the air sac bottom wall and patient's posterior wall of the throat. At present drawings the second drainage tube 102 is on the left side of the drainage passage 100. The fluid cumulated under the bottom fluid groove 77 can easily enter into the second drainage tube distal opening 108 and then to be suctioned out by an outside suction device. In an alternative embodiment, second drainage tube 102 can be omitted.

FIG. 9d, in another alternative embodiment, the second drainage tube 102 and the distal drainage tube 109 are omitted. The drainage passage is configured as a single hollow drainage tube, called central drainage tube 111. A central drainage tube 111 has replaced the drainage passage 100. The central drainage tube 111 has a central drainage tube proximal opening 114 and a central drainage to be distal opening 113. The central drainage tube distal opening 113 terminates shortly after passing beneath the air tube distal end 29. The central drainage tube distal opening 113 is totally separated from the air tube distal opening 29 by the air sac bottom wall. Otherwise the configuration of the air tube lumen, outside shape of the air tube, the air sac and tongue lifting means, and the pulling unit 60 are all completely the same as described previously described. But in the FIG. 9d, the pulling unit 60 is not shown for the purpose of a better showing the central drainage tube 111. Viewing from the bottom surface of the air sac will have a longitudinal central vacant area, called fluid hood 115, which used to be occupied by the distal drainage tube 109. The distal end of the fluid hood 115 is an entrance for fluid from the upper esophagus, called fluid entrance 112. When the patient is in supine position, the fluid from the upper esophagus passes through the fluid entrance 112 and enters the area under the fluid hood 115 at where the fluid cumulate and then flow into the central drainage tube 111 via the central drainage tube distal opening 113 to be sanctioned out. An outside suction device can be connected to the central drainage tube proximal opening 114 as usual. The expanding and un-expanding of the sac airflow channel 78,79 by inspired gases during a respiratory circle can have some propelling effect to move the fluid on surface of the surrounding tissues into area under the fluid hood 115 when a patient is in a supine position.

### The Second Embodiment

In second embodiment, if an element is configured with the same function as in the first embodiment, the same name and number label will be assigned. If an element has same function but configuration is not completely same as in the first embodiment, then a same name but a different number label will be assigned.

The second embodiment has the same configuration of the tongue lifting means, the pulling unit, and the drainage system as in the first embodiment. But few new alternative designs have been configured, which are a distal airflow tunnel, divergent plate 28a, sac lifting plate 99a, pulling stick 66a, and band anchor 65a. Structures of the upward ramp 22 and the downward ramp 23 have been deleted. The rest of configuration of the air sac will be same as in the first embodiment, FIG. 2a, 3a, 6b, 8a, 8b, 12, 13, 14a, 14b, 14c.

The air tube outside shape in cross sections in the air tube distal segment 15 is same as in the first embodiment also preferably configured as horizontal rectangle or alike, and is also surrounded by same air tube bottom wall also called posterior wall 17, same air tube top wall 19, and same right and left air tube side walls 16. And inside of the air tube lumen shape will be divided into three channels, starting from the Y point 18, same as in the first embodiment. However, at in the air tube distal segment 15, a distal airflow tube 38 or a distal airflow channel 15a and divergent plates 28a have been added.

The top tube airflow channel is becoming a tube-like structure with completely physically surrounding walls after separating from the right and left tube airflow channel 32, 31, and is extending further distally into the air sac, called the distal airflow tube 38. The distal airflow tube 38 is a length of a tube structure and is preferably configured as a rectangle-like shaped tube with an upper wall, two side walls and a bottom wall. The distal airflow tube 38 can also be configured as square, trapezoid, ellipse or semi-ellipse, circle or semi-circle or other geometric shape or combinations, or its shape can be changed over its course.

The distal airflow bottom wall 29a is a sheet of thin plastic material with similar property as the air tube distal segment 15, preferably with more flexibility. Distal airflow bottom wall 29a starts and extends from the drainage passage top wall 35 in the air tube distal segment 15 toward distally. Its right and left side edges couple with inside of right and left air tube side wall 16 and therefore to constitutes a complete and independent bottom wall of the distal airflow tube 38. The starting point of the distal airflow bottom wall 29a is physically and completely separating point between the between the distal airflow channel 38 and right and left tube airflow channels 32, 31, called split point 18c. The distal airflow tube 38 is a continuation of the top tube airflow channel 30. Two side walls of the distal airflow tunnel 38 are continuation of the air tub side walls 16 but become narrower, called right and left distal airflow side wall 29b. The upper wall of the distal airflow tube 38 is continuation of the air tube top wall 19. All these surrounding walls extend distally same length and stop at same time to constitute the most distal opening of the entire air tube, called air tube distal opening 29, or air tube distal edge 29. The right and left tube airflow channels 32, 31 will terminate at or near the split point 18c and smoothly transition to the right and left sac airflow channels 78, 79 respectively.

Alternatively, at the air tube distal opening 29, a distal portion of the top wall of the distal airflow tube 38 can be deleted structurally, rest of the top wall is called incomplete top wall 29d, FIG. 3a, 6b. Therefore, the air tube distal opening 29 becomes an irregular shaped and enlarged. This configuration is called distal airflow channel 15a. The distal airflow channel 15a will have same distal airflow bottom wall 29a, right and left distal airflow side walls 29b as the distal airflow tube 38 but have a incomplete top wall 29d. The distal opening of the distal airflow channel 15a is also called the air tube distal opening 29, or interchangeably called the air tube distal edge 29.

The distal airflow tube 38 and the distal airflow channel 15a both preferably are configured to bend up in a smooth curve therefore the distal airflow bottom wall 29a forms a sharp angle with the distal drainage tube 109, or in other words with sac central protruding surface 39. This angle is called split angle 18b, or interchangeable called split space 18b, FIG. 3a, 6b, 14a, 14b, 12c1, 12c2. The distal airflow tube 38 and the distal airflow channel 15a both are configured to guide airflow and deliver the airflow closely toward the vocal cords opening. The both are collectively called the distal airflow tunnel.

The tube -shaped distal airflow tube 38 is also configured to create a focused, restricted, or jet-like airflow and direct this airflow to the vocal cords opening during intermittent positive pressure ventilation (IPPV). In contrast the distal airflow channel 15a is also to create a focused, restricted or jet-like airflow but is able to deliver the jet-like airflow in a wider spectrum toward vocal cords opening due to its enlarged distal opening.

The split space 18b is configured to be changeable for its angle degree. Before being inserted into a patient or animal's throat, the split space 18b is a small space, during insertion of the device because the operator need to bend the air tube distal segment 15 and the air sac 80, therefore the distal airflow bottom wall 29a will contact or even press on the distal drainage tube top wall or the sac central protruding surface 39. The distal airflow bottom wall 29a will be forced to contact the distal drainage tube top wall or the sac central protruding surface 39 therefore the split space 18b become smaller or disappear. After the device is inserted in a proper position, the operator's hand remove the bending force, the air tube distal segment 15 and the distal airflow bottom wall 29a will resume their original angularity because of the elasticity of the materials of the device, therefore the split space 18b become bigger again. This ability of changing degree or space of the split space 18b is configured to facilitate the device insertion, FIG. 14a, 14b.

In a preferred embodiment, in the middle of the distal and in long axis of the device the distal airflow bottom wall 29a is configured to have a groove like longitudinal depression, called central groove 29c. The central groove 29c is like a guide groove for a stylet, and is to guide a stylet easily toward the vocal cords opening in a case of an intubation is needed after the device inserted in a proper position. And also because the distal airflow bottom wall 29a is curved up toward the vocal cords opening and the distal airflow tunnel opening or edge 29 is in proximity with the vocal cords opening, an operator can use a stylet to follow the central groove 29c and to push the stylet into the vocal cords opening during the intubation.

In one embodiment, a right and a left divergent plate 28a have been configured, FIG. 14a, 14b, 14c. Each of the divergent plate 28a is trapezoid or rectangle or ellipse or semi-ellipse like shaped thin bendable plates with its width substantially narrower than width of the left tube airflow channel 31 or right tube airflow channel 32 therefore airflow can pass through right or left side of the divergent plate 28a toward distal direction. The divergent plate 28a can be configured as many other different geographic shapes, but preferably is configured as trapezoid shape with its wider side, called divergent plate upper edge 28b, connected with proximal edge of the distal airflow bottom wall 29a. The narrower side of the divergent plate 28a is suspended in the air, called the divergent plate lower edge 28c. The right and left divergent plate lower edge 28c will not touch the air tube bottom wall 17 when are in naturel position and are not being pushed by a force. Alternatively the divergent plate 28a is extension of the distal airflow bottom wall 29a therefore the divergent plate upper edge 28b would be omitted. Therefore in this configuration, a fixed amount airflow will be diverted into the distal airflow tunnel.

The divergent plate 28a are diagonally or semi-vertically positioned inside the right or left tube airflow channel 32, 31. The divergent plate 28a are design to be narrower than the right or left tube airflow channel 32,31 and are narrow enough to allow enough amount airflow pass around the right or left side of the divergent plate 28a. The amount of the passed airflow allow can be predetermined during manufacture therefore divergent plate 28a would only “block” a portion of the airflow.

Consequently the divergent plate 28a will “block” a certain amount of airflow in the right or left of the tube airflow channel 32, 31 and divert these “blocked” airflow into the distal airflow tube 38 or the distal airflow channel 15a. However, a maximum amount of the airflow can be divergent into the distal airflow tunnel can be predetermined during manufacture. The larger total area of the divergent plate 28a, the more airflow will be “blocked”, especially in IPPV. The more airflow is diverged into the distal airflow tunnel during intermittent positive pressure ventilation or during spontaneous ventilation, the more airflow pressure will be increased inside of the distal airflow tunnel. Therefore, a focused, restricted or a jet-like airflow is created especially during intermittent positive pressure ventilation. The pressure increased focused or restricted or jet-like airflow will be directed to the vocal cords opening. Additionally, the distal opening of the distal airflow tunnel is designed to near the vocal cords opening; therefore the present device can be effectively used to perform mechanic ventilations.

In another embodiment, the divergent plate upper edge 28b is configured similarly like indentation line 25 which is “hinge” like or thinner linear structure and is readily to be bent under a force. The right and left side 28b have similar “hinge” function. During inspiration phase of intermittent positive pressure ventilation, an increased inspiratory airflow pressure inside of the right and left tube airflow channel 32, 31 will push the divergent plate 28a swinging distally by using the divergent plate upper edge 28b as bending hinge, therefore the divergent plate 28a will become more vertical position and divergent more airflow flowing into the distal airflow tunnel. Ability of how much force to bend the divergent plate upper edge 28b can be pre-determined. Even though the divergent plate upper edge 28b is readily bendable, the divergent plate upper edge 28b is also configured strong enough only to be bent by predetermined amount of pushing force, and also is strong enough to resume the divergent plate 28a original position after the airflow force stopped during expiration phase of IPPV. During the expiration, the divergent plate 28a can resume their original positions to let airflow freely venting out. Therefore, the divergent plate 28a is like self-adjustable valve.

A control mechanism of swing distance or moving distance of the divergent plate 28a has been configured and can be designed in different ways. However, the concept is within protection of present invention.

To control the moving distance, a divergent plate stopper is configured on the air tube bottom wall 17, or on the right or left drainage passage side wall 36, or on the inside of the right or left air tube side wall 16, all called the divergent plate stopper 28d FIG. 14a, 14b. The divergent plate lower edge 28c is a free edge suspending in air if not being pushed by an incoming positive pressure airflow inside the right or left tube airflow channel 32, 31 and stay a predetermined distance from the divergent plate stopper 28d. The divergent plate stopper 28d will stop distal movement of the divergent plate lower edge 28c once the divergent plate lower edge reaches the divergent plate stopper 28d in predetermined moving distance. Alternatively, the right or left divergent plates 28a can be simply configured longer enough to reach and be stopped by air tube bottom wall 17 without configurations the divergent plate stopper 28d. No matter by using the divergent plate stoppers 28d or the air tube bottom wall 17 as a tool to stop the movement of the divergent plate 28a, they are collective called the divergent control means.

If a positive airflow pressure to push the divergent plate 28a is not very strong and can only swing the divergent plate lower edge 28c half way to reach the divergent control means, the less amount of the airflow will be divergent by the divergent plate 28a into the distal airflow tunnel. Therefore the “selective” bendability of the divergent plate upper edge together divergent control means have established self-adjustable capability for diverging a predetermine amount of the airflow, or an automatic self-adjusted airflow division mechanism, called airflow self-control mechanism. The airflow self-control mechanism can “automatically” choose to divide and guide more or less amount incoming airflow inside of the right and left tube airflow channel 32, 31 into the distal airflow tunnel according the airflow pressure during intermittent positive pressure ventilation.

When the device is used for a spontaneous respiration, during inspiration airflow in from proximal to distal along the air tube lumen is due to a negative inhale pressure generated by a patient or animal, the negative inhaled pressure is much less powerful and may not strong enough to move the divergent plate 28a significantly.

The distal airflow channel 15a has an enlarged the air tube distal opening 29 and can deliver the pressured focused or jet-like airflow toward the vocal cords opening in a wider angle. In contrast, the distal airflow tube 38 has narrow the air tube distal opening 29 and can direct pressurized focused or jet-like airflow towards the vocal cords opening more precisely manner, during IPPV.

The upward ramp 22 inside of the air sac in the first embodiment is to guide the airflow toward the vocal cords opening, the upward ramp central groove 21 is to guide a stylet into the vocal cords opening. The both function have been replaced by the distal airflow tunnel in the second embodiment. The rest of air sac configurations in the second embodiment are same as in the first embodiment.

An alternative design of the tongue lifting means, called sac lifting plate 99a has been configured. When being pulled, the sac lifting plate 99a can lift the tongue base in the same fashion as the lifting plate 15 or the bar lifting assembly 95. And the sac lifting plate 99a can also be constructed in the first embodiment in replacing function of the lifting plate 15 or the bar lifting assembly 95. The sac lifting plate 99a is a thin plate made with plastic, or silicon or other materials with similar properties. The sac lifting plate 99a fuses with right and left the sac wall proximal edge 88 and close the gap between the sac wall proximal edge 88. The lower edge of sac lifting coupling to the air tube top wall 19 via the indentation line 25 in same fashion as in the first embodiment. The right and left pulling bands 66 or the pulling stick 66a will attach to the sac lifting plate 99a via the right and left band distal ends 68, and pull up the sac lifting plate 99a in same fashion as the lifting plate 15 or the bar lifting assembly 95. In this configuration the sac wall 81 would be configured more flexible and foldable. When the sac lifting plate 99a is in relaxed un-pulled position, the sac lifting plate 99a will be manufactured as “pushed” down position in which the sac lifting plate 99a is positioned more horizontally than vertically positioned. The sac lifting plate 99a is semi-facing down to the sac bottom wall. Therefore, the sac lifting plate 99a will make the right and left sac walls bend or fold partially into the air sac chamber. When the sac lifting plate 99a is pulled up by the pulling band 66, the sac lifting plate 99a will stretch the inward strips 86 and the sac wall descending edges 82 intense and same time pull up the right and left sac walls 81 and therefore open up the sac top opening. The sac lifting plate 99a also can be used in the first embodiment in combination with other elements.

The pulling unit 60 can be configured same as in the first embodiment. However, an alternative of the pulling stick 66a are configured with same function of the pulling band 66. Also the pulling anchors 65a have been configured as alternative of the band sliding pad 65.

At the right and left air tube side wall 16, or the air tube bottom wall 17, or air tube top wall 19, or the corner of the right or left air tube side wall 16 and the air tube bottom wall 17 or corner of the right or left air tube side wall 16 and the air tube top wall 19, one or more pair of prominent or tubercle-like structures are configured on right and left symmetrically along the air tube, called band anchors 65a. The band anchors 65a can be shaped as a smooth protruding tubercle with a groove beneath, or a face down semi-opened hook. The band anchors are used as anchor to allow the right and left pulling bands 66 or the pulling sticks 66a to move back and forth easily, and to convey the pulling force to the right and left band distal end 68.

The pulling stick 66a is a curved shape with semi-rigidly thin rod, and the curve can be bent further or straighten under a force in certain extent. The curvature can be variable. A portion curvature of the pulling stick 66a is preferably manufactured with similar curvature of the air tube distal segment 15. The right and left pulling bands 66 or the pulling sticks 66a can also use the right and left band anchors 65a as anchors to move back and forth and to be kept on right and left side of the air tube. The distal end of the right and left pulling stick 66a are also attach to the tongue lifting means same way with same function as described in the first embodiment, so also called the right and left band distal ends 68. The pulling stick 66a is an alternative design of the pulling band 66, therefore all concept and claim of the pulling bands 66 will be fully apply to the pulling stick 66a.

Further alternatively, the pulling stick 66a can be configured as a portion as semi-rigid stick and a portion as more flexible bans or string, or curved spiral like. These variable configurations can perform similar functionalities as the pulling band 66 and belong to variation of same concept, are all protected in the present invention.

All above those alternative designs, such as but not limited, the band anchor 65a, the sac lifting plate 99a and the pulling anchor 66a are to be optionally used to replace their counterpart's function in the first embodiment.

The drainage system in the second embodiment would be same as in the first embodiment and can choose or omit one or more drainage tubes or different combinations.

### The Third Embodiment

Same as previously, if an element is configured same with same function as in the first embodiment, the same name and number label will be assigned. If an element has same function but configuration is not completely same as in the first embodiment, then a same name but a different number label will be assigned.

The third embodiment will comprises an air tube, an air sac, the tongue lifting means and the pulling unit 60 without any drainage tube configuration which will simplify the manufacture process.

Referring to FIG. 15a, 15b the air tube include an air tube proximal segment 119a and an air tube distal segment 119b and is a hollow tube as no structure inside the air tube lumen 120. Therefore, the outside shape of the air tube is same as inside. The air tube has proximal opening 20 and an air tube distal opening 117 or the air tube distal edge 117. The tongue lifting means and the pulling unit 60 are completely same as in the first and the second embodiments. However, the FIG. 15a only uses the lifting plate 51 as a representative for the tongue lifting means. And all three designs of the tongue lifting means are couple to the top wall of the air tube distal segment 15 via the indentation line 25 in the same fashions as in the first and second embodiments. The pulling unit 60 and hook or hooks 11 are configured in the same as in the first and second embodiment, FIG. 15a.

A distal portion of the air tube distal segment 119b is configured to extend distally inside the air sac 119. This portion of the air tube distal segment 15 is a tube like structure, called distal airflow segment 118. A cross section area of the distal airflow segment 118 can be same as the air tube distal segment 119b, but preferably is smaller. The smaller cross section area is more likely to create a focused, directed or a jet-like airflow same principle as the distal airflow tunnel in the second embodiment. The distal airflow segment 118 bends up as a smooth curve and has a distal opening which is the air tube distal opening 117, also called the air tube distal edge 117.

The air tube distal opening 117 is the most distal end of the whole air tube same concept as in the first and second embodiments. The preformed bend-up shape of the distal airflow segment 118 is configured to guide airflow toward the vocal cords and to deliver the airflow close to the vocal cords opening for the same purpose as the distal airflow tunnel in second embodiment.

Proximally 0.2-3 cm from the air tube distal edge 117, a pair of hole or few pair of holes have been configured on the right and left side wall of the distal airflow segment 118 or right and left side of the bottom wall of the distal airflow segment 118, or combination of the both, called air holes 116, FIG. 17b. The air holes 116 can be different geometric shapes, rectangle, triangles, ellipse or circle, preferably circles. Diameter of these holes can be from 0.1 cm to 2.5 cm. Because the distal airflow segment 118 has been preformed bend-up, the up-curve shape can make the airflow not easily flow into the right and left sac airflow channel 118a, 118b. The air tube holes 116 are configured to let a portion of incoming airflow from the air tube easily flow into right and left sac airflow channel 118a, 118b. The airflow entering the right and left sac airflow channel 118a, 118b are to inflate the right and left sac airflow channel same mechanism as described in the first and second embodiments. Therefore, the air holes 116 at distal end area the air tube are served as a tool to divide the airflow. Please note that just like in the first and second embodiments, even though an incoming airflow is divided into three airflows, but these three airflows once get out from there physically structured airflow channel, they are still able to meet and mix inside of the air sac. Even though design or mechanism of how dividing the airflow by the air holes is different from the first and second embodiment, the essential concept of dividing incoming one airflow into three directions, one toward to vocal cords opening, other two toward to the right and left sac airflow channel respectively, is same and is within scope of protection of the present invention.

However the air sac 119 is configured differently. In the third embodiment, the airflow inside the air sac may or may not be divided. In one design, the sac central protruding surface 39 is replace by a central longitudinal belt shaped thickening protruding area, called sac central thickening zone 39a. Along right and left side of the sac central thickening zone 39a a right sac airflow channel 118a and a left sac airflow channel 118b have been configured to function similarly as the right and left sac airflow channel 78, 79 in the first and second embodiment. However, the right and left sac airflow channel 118a, 118b will be configured shallower than previously described the right and left sac airflow channel 78, 79. Alternatively, the sac central thickening zone 39a can be omitted therefore the sac airflow channels 118a, 118b would be omitted too.

The distal portion of the air sac can be configured to have a sac distal pocket 123. On top of the sac distal pocket 123 there is also a thinner top film or plate, called sac distal pocket cover 121. The distal end of the air sac 119 is configured as a plug shaped with a round enclosed end to block the upper esophagus opening, called sac distal plug 122. The sac distal pocket cover 123 is configured similar as the sac distal pocket 99 in the first and second embodiments. Similarly the inspired airflow enters inside the sac distal pocket 123 can push up the sac distal pocket cover 121 to form a seal with the posterior laryngeal wall.

A portion of inspired airflow during inspiratory phase of a respiratory circle will flow into the air sac 119 and a portion of the airflow will flow into the sac distal pocket 123 which can expand the sac distal pocket 123 therefore to further enhance the seal with the upper esophagus in addition to a plugging function of the sac distal plug 122. The sac central thickening zone 39a will strengthen the air sac 119 and make the device easier to be inserted into throat and not easily twisted or folded. The rest of element of the air sac, such as the sac wall descending edges 82, the inward strip 86, the sac wall holding zone 85 the sac vertical attachment edge 71, the sac horizontal attachment edge 72 and the sac airflow channel side wall 76 have same configurations as in first and second embodiments.

In an alternative design, the air holes 116 can be omitted to further enhance the increasing airflow pressure inside of the distal airflow segment and deliver more airflow toward the vocal cords opening.

At the bottom wall of the distal airflow segment 118, a groove like indentation is configured to guide a stylet for an intubation same purpose as the upward ramp central groove 21 and the central groove 29c. Therefore, all three grooves are collectively called stylet groove.

As previously mentioned, all elements of all three embodiments can easily be exchanged and create a new combination of design, but these new configurations are based on same concepts and are completely protected by the present invention.

### OPERATIONAL EXAMPLE

The present device is to be used as a supraglottic airway device for spontaneous or intermittent positive pressure ventilation, and can be used as a conduit to guide a stylet into the vocal cords opening during an endotracheal intubation. The device is first prepared for insertion. Outside surface of the distal and middle portion of the device are well lubricated.

An operator will use one hand to open a patient's or animal's mouth by using a standard technique. The other hand holds the device and inserts the sac distal edge 87 into a patient or animal's against the roof middle line of the mouth. The operator continue to push the device and same time make the device bend more anteriorly for more easily sliding down around the curvature of the back of the tongue. When the device is inserted in a certain depth, the operator's hand will feel increased resistance and is difficult to advance more. This is indication that the device is in a proper position. The depth marks on the air tube wall can also be used as a reference.

After the device is in the proper position, an operator will start pulling the band tail 63 of the pulling unit 60. The pulling band 66 will pull up the lifting plate 51 or its alternative design of the bar lifting assembly 95 or the sac lifting plate 99a. The plate distal edge 53 of pulled the lifting plate 51 will touch the tongue base and be bent to form seal with the tongue base. The epiglottis elevation plates 58 or the central epiglottis elevation plate 59 will also be raised and most likely touch and push the epiglottis up to further open the laryngeal inlet. The plate distal edge 53 also will push up the right and left inward strip 86. At same time the right and left plate side edges 52 will further touch the inner surface of the sac wall 81 and push right and left sac wall 81 aside to open up more of the sac top opening 70. The pulling bands 66 or the right or left pulling sticks 66a will pull the right and left sac wall proximal edge 88 further apart. Then the operator can dock one of the band holes on the hook 11 or hooks 11 so the operator can free one hand. And then operator connects respiratory equipment to the air tube proximal opening 20.

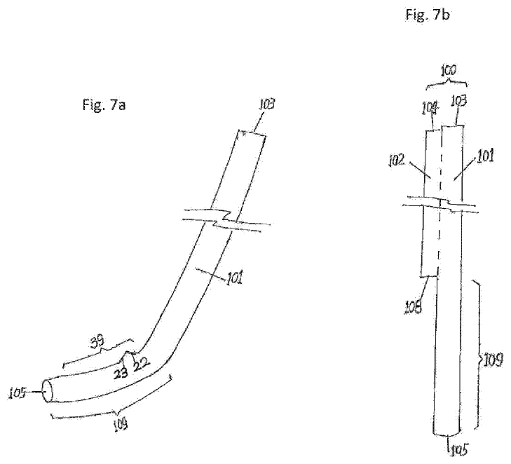

The first and second drainage tube's proximal openings can be connected suction device separately or their common outlet as needed. In a long surgical case, after the device is inserted and the above processes are completed, the operator should reexam and adjust the tightness of the pulling bands by docking different band hole on the hook 11 according each specific patient condition. This can further limit pressure on the pharynx tissue and the tongue base, and diminish a chance of tissue damage. A proper sized oral gastric tube can be passed through the first drainage tube 101 and the distal drainage tube 109 into esophagus and into stomach to suction out the fluid and small particles. If the central drainage tube 111 is configured in the alternative design FIG. 7b, an outside suction device can be connected with the central drainage tube proximal opening.

If an endotracheal intubation is needed, a commercially available stylet with a bent tip, such as, not limited, a Bougie, can be used and an operator just follow a currently standard maneuver to do intubation. However in the present invention, the central groove 29a or the tube groove 118c in the third embodiment can provide a guide for the stylet toward the vocal cords opening. Any currently available fiber-optic-scope-probe can be inserted into proximal opening of the air tube and advance all the way to the air tube distal opening 29 or the distal airflow outlet opening 117 in the third embodiment to help intubation under vision.

What is claimed is:

1. An airway device to be inserted into a patient's or animal's throat and by an operator's hand pulling force to cause deformation of the device before performing its function comprising: an air tube; an air sac; a tongue lifting means; a pulling unit and a drainage system,
Wherein the air tube includes an air tube lumen with a proximal opening and a distal opening and having an air tube proximal segment and an air tube distal segment, wherein the air tube is configured to having a bottom wall, a top wall and right and left side walls to form an air tube outside shape, wherein shape of cross section of the air tube lumen varies along the air tube length, wherein a drainage system is coupled to the bottom wall of the air tube lumen and reshape the air tube lumen as a top tube airflow channel, a right tube airflow channel and a left tube airflow channel therefore a configuration of inside air tube lumen is to divide the airflow and guide the airflow, and, wherein the air sac includes a sac bottom wall, a left sac wall and a right sac wall an air sac chamber and sac top opening, wherein the sac bottom wall has a sac central protruding surface, a right sac airflow channel, a left sac airflow channel, wherein the right and left sac airflow channel are configured to position on the right or left side of the sac central protruding surface, wherein the air sac has a right and a left sac proximal attachment respectively, wherein the right and left sac proximal attachments are configured to attach the air tube distal segment, wherein the right tube airflow channel smoothly adapts and continues with the right sac airflow channel, wherein the left tube airflow channel smoothly adapts and continues with the left sac airflow channel, wherein the right and left right and left sac airflow channel extend distally to form the sac distal pocket, and
wherein the tongue lifting means is configured to be pulled by the pulling bands, wherein the raised tongue lifting means is configured to push up the tongue base and form seal with the tongue base, the pulling band is configured to be pulled by an operator hand to generate a mechanical deformation force to lift the tongue base.

2. The device of claim 1, wherein the materials forming the right and the left sac airflow channel and the sac distal pocket are elastic, compressible and can be expanded by inspired airflow during intermittent positive pressure ventilation, wherein the right and the left sac airflow channel and the sac distal pocket can be expanded by inspired airflow during the inspiratory phase of the respiration cycle.

3. The device claim 2, wherein the sac distal pocket has configured to have a sac distal pocket cover, wherein the sac distal pocket cover is configured to be constructed more flexible and to be pushed up by the pressured air inside the sac distal pocket to seal with posterior wall of larynx during the inspiratory phase of the respiratory cycle.

4. The device of claim 1, wherein at distal to the air tube distal opening the sac central protruding surface is configured to bend up to from an upward ramp and bend down to form a downward ramp, wherein the upward ramp is to help guide the top tube airflow align to vocal cords opening, and a stylet tip into the vocal cords opening.

5. The device claim 1, wherein the right and the left sac wall include a right and a left sac wall descending edges, a right and a left inward strips, and a right and a left sac wall proximal edges, wherein the right and left sac wall descending edges are configured to continue with the right and left inward strips, therefore when the right and left inward strips are pulled the right and left sac wall descending edges are configured to be pulled and stretched tense to prevent the right and left sac airflow channel collapse.

6. The device of claim 5, wherein the right and left sac wall are configured to have a right and a left sac wall holding zone, wherein right and left sac wall holding zone are configured to be perpendicularly attached to the top wall of the air tube distal segment, wherein the right and left sac wall holding zone are configured to structurally continue with the right and the left inward strips, and wherein when the lifting means is pulled up the right and left sac wall holding zone will create a resistance to the rising tongue lifting means and at same time the right and the left sac wall holding zone will be pushed by the raised tongue lifting means, therefore the right and the left sac wall holding zone will pull the right and the left inward strip.

7. The device claims 5, the right and left sac wall descending edges are configured to be significantly vertical in related with the sac distal pocket and the right and left inward strips, therein the right and left sac wall descending edges are configured to encounter the right and left side of laryngeal inlet structures and create the resistance for the operator to stop advance further during the device insertion.

8. The device of claim 6, wherein the tongue lifting means is configured to couple to the top wall of the air tube distal segment, wherein the tongue lifting means is configured to be pulled up diagonally by the pulling bands, wherein the tongue lifting means can be configured as a lifting plate, wherein the tongue lifting plate has lifting plate distal edge, a right plate side edge and a left plate side edge, wherein when the lifting plate being pulled up the plate distal edge will push up the right and left inward strip and form seal with the tongue base ,and the right and the left plate side edges of the lifting plate are configured to push the right and the left sac wall further aside to form seal with the pharyngeal walls and to further open up the sac top opening, wherein the right and the left plate side edges are configured to bent to form more contact area with the right and left sac walls.

9. The device claim 8, wherein the tongue lifting plate is configured to have a central epiglottis elevation plate or two epiglottis elevation plates on the lifting plate distal edge, wherein the central epiglottis elevation plate and the two epiglottis elevation plates can contact and lift the epiglottis when the lifting plate is pulled up diagonally by the pulling bands.

10. The device claim 1, wherein the lifting means is configured as bar lifting assembly, wherein the bar lifting assembly has been configured to be pulled by the pulling unit 60 therefore to push up the tongue base, wherein the bar lifting assembly has two lifting bars, a central fold between the two lifting bars and two lateral folds, wherein the two lateral folds are configured to connect the two lifting bars with the right and left sac wall holding zones respectively, wherein the two lifting bars are to be pulled up by the pulling bands, wherein when the pulling unit 60 pulls up the bar lifting assembly diagonally the central fold and two lateral folds will unfold.

11. The device claim 8, wherein between the lifting plate and the right and left sac wall holding zones an enhanced sealing means has been configured to create seal between the lift plate and the right and the left sac wall holding zones.

12. The device claim 8, the pulling unit 60 are configured as one or more long bands traveling along with the air tube at above the air tube, below the air tube or on right and left sides of the air tube, or combination thereof, wherein the pulling bands have one or more distal ends to attach to the lifting means and one or more proximal ends, wherein the pulling bans have one or more band hole, wherein the distal ends are firmly attached to the tongue lifting means and the proximal ends are for operator's hand pulling and are to be docked at a certain predetermined pulling distance.

13. The device of claim 12, wherein the right and the left pulling bands are configured to touch the right and left sac wall proximal edges, wherein when the right and the left pulling bands are pulled tense, the tightened the right and the left pulling bands will push the right and left sac wall proximal edges further apart.

14. The claim device 1, wherein the drainage system includes a drainage passage and a distal drainage tube, wherein the drainage system configure to divide the airflow inside of the air tube lumen and airflow inside the air sac and to drain fluid from the esophagus.

15. The device claim 14, Wherein the drainage passage can be configured as a central drainage tube, wherein the central drainage tube is a hollow tube and has a central drainage tube distal opening and a central drainage tube proximal opening, wherein the central drainage tube distal opening is terminated after passing beneath the air tube distal opening, wherein beneath the sac central protruding surface is configured to have a fluid hood, wherein the central drainage tube distal opening is configured to drain the fluid cumulated under the fluid hood.

16. An airway device to be inserted into a patient' or animal's throat is comprising an air tube, an air sac; a tongue lifting means; a pulling unit; and a drainage systems;
wherein the air tube is a hollow tube having an air tube proximal segment and an air tube distal segment, and having a proximal opening and three divided distal openings, wherein one of the three divided openings on the top extends further distally to form a distal airflow tunnel, wherein the other two divided airflow openings are configured to transition to as a left tube airflow channel and a right tube airflow channel, wherein the distal airflow tunnel has an distal air tube opening, and
wherein the drainage systems is configured to enter the air tube lumen at Y point and reshape the air tube lumen and separate with the air tube lumen at the spit point, wherein at the split point, the air tube lumen is completely divided incoming airflow into three airflows, wherein of the three airflows is guided to the vocal cords opening, other two airflows are guided into the air sac, and wherein the drainage system is also configured to drain the fluid from the esophagus, and
the air sac includes a sac bottom wall, a left sac wall and a right sac wall an air sac chamber and sac top opening, wherein the sac bottom wall has a sac central protruding surface, a right sac airflow channel, a left sac airflow channel, wherein the air sac has a right and a left sac proximal attachment respectively, wherein the right and left sac proximal attachments are configured to attach to the air tube distal segment, wherein the right sac airflow channel is configured to smoothly adapt and continues with the right sac airflow channel, wherein the left tube airflow channel smoothly adapts and continues with the left sac airflow channel,
wherein the tongue lifting means is configured to be pulled up by the pulling unit, wherein the raised tongue lifting means is configured to push up a tongue base, the pulling unit is configured to be pulled by an operator's hand therefore the tongue base lifting is executed by the operator generating a mechanical force on the pulling unit.

17. The device claim 16, therein the distal airflow tunnel has a distal airflow bottom wall, wherein the distal airflow bottom wall is configured to form a split angle at the sac central protruding surface, 39, wherein the split angle is changeable under a force to facilitate the device insertion.

18. The device of claim 17, further comprising a right and a left divergent plate configured in the right and left tube airflow channel, wherein the right and left divergent plate extend diagonally into the right and left tube airflow channel, wherein the right and left divergent plate are configured to divergent more airflow from the right and left tube airflow channel into the distal airflow tunnel.

19. The device of claim 18, wherein the divergent plate has a divergent plate upper edge and a divergent low edge, wherein the divergent plate upper edge is configured to be bendable by preconfigured amount of airflow pushing force generated by an increased airflow pressure during intermittent positive pressure ventilation therefore to establish a self-adjustable capability to enable an changeable amount of airflow to be divergent into the distal airflow tunnel according to the airflow pressure changes during intermittent positive pressure ventilation, and wherein two divergent plate stoppers have been configured in the right and left tube airflow channel to control moving distance of the right and left divergent plate respectively therefor to establish an airflow self-control mechanism.

20. The device of claims 15, wherein the distal airflow tunnel is configured as distal airflow channel, wherein the distal airflow channel has an enlarged distal opening, wherein the enlarged distal opening of the distal airflow channel is shaped to deliver an airflow substantially across the entire vocal cords opening.

21. The device of claim 20, therein the distal airflow tunnel is configured as a distal airflow tube, wherein the distal airflow tube is configured as a tube structure to extend close to vocal cords opening, wherein the distal airflow tube is configured to direct the focused airflow at vocal cords opening during.

22. The device of claim 21, wherein a central groove has been configured on the distal airflow bottom wall, wherein the central groove is to guide a style easily into the vocal cords opening during intubation.

23. The device of claim 15, wherein one of the tongue lifting means includes a sac lifting plate, wherein the sac lifting plate is connected to the right and left sac wall, wherein when the sac lifting plate is pulled up by the pulling unit, the right and left sac wall will stand up and open up the sac chamber.

24. The device of claim 15, further comprising one or more band anchors, wherein one or more band anchors are coupled to the air tube's right side, left side, upper edge, lower edge, or combination thereof, one or more band anchors are configured to enable the pulling and back and forth when an operator is pulling and releasing the pulling unit.

25. An airway device to be inserted into a patient' or animal's throat comprise an air tube, an air sac; a tongue lifting means and a pulling unit; wherein a mechanical force is needed to pull up the tongue lifting means therefore to push up the tongue base,
wherein the air tube is a hollow tube with an air tube proximal opening and an air tube distal opening and having an air tube proximal segment and an air tube distal segment, wherein the distal portion of the air tube distal segment extend inside the air sac to form a distal airflow segment, wherein the distal airflow segment is to direct an airflow at the vocal cords opening, and
wherein the air sac includes a right and left right sac wall, an air sac bottom wall, a sac distal pocket, an air sac chamber and a sac top opening, wherein the sac wall includes a right and a left inward strip 86, a right and a left sac wall descending edges 82, wherein the air sac includes a right and a left sac proximal attachment to couple to the air tube distal segment, wherein the right and left sac wall are configured to have a right and a left sac wall holding zone 85, wherein the right and left sac wall holding zone are coupled to the top wall of the air tube distal segment, and
wherein the tongue lifting means is configured to be pulled by the pulling unit, wherein one more pulling bands are configured to be pulled and convey the pulling force to the tongue lifting means, wherein the raised tongue lifting means is configured to push up the tongue base and form seal with the tongue base, wherein the raised lifting means is configured to push the side wall of the air sac aside and push up the right and left inward strip, wherein the raised lifting plate is configured to push the right and left sac wall holding zone therefore to cause pulling the right and left inward strips and the descending edge, wherein after these deformation changes, the right and left inward strips, the right and the left descending edges will form seal with a different area of surrounding tissues, wherein when pulling bands are stretching and also directly lift the tongue base in addition to the action of pulling up the tongue lifting means,

26. The device of claim 25, wherein the distal airflow segment is configured in up-curved shape tube-like structure and has a complete surrounding wall, wherein on the distal airflow segment's right and left side or bottom side of the distal air tube segment, a pair of air hole or multiple pairs of air holes have be configured, wherein air holes are configure to allow a portion of an incoming airflow inside of the air tube lumen into right side of the air sac.
